(19) United States
(12) Patent Application Publication
(10) Pub. No.: US 2006/0030535 A1
(43) Pub. Date: Feb. 9, 2006

(54) Controlled modulation of the pharmacokinetics and biodistribution of aptamer therapeutics

(76) Inventors: Judith M. Healy, Lexington, MA (US); Markus Kurz, Newton, MA (US); Thomas Greene McCauley, Cambridge, MA (US); Kristin Thompson, Arlington, MA (US); Charles Wilson, Concord, MA (US); Dorothy J. Margolskee, Upper Montclair, NJ (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/075,648

(22) Filed: Mar. 7, 2005

Related U.S. Application Data
(60) Provisional application No. 60/550,790, filed on Mar. 5, 2004.

(51) Int. Cl. A61K 48/00; A61K 9/14

(52) U.S. Cl. 514/44; 424/486

(57) ABSTRACT

Materials and methods are provided to modulate, in a controlled manner, the pharmacokinetic and biodistribution properties of nucleic acid aptamers, and to enhance their safety and efficacy properties as therapeutic agents.

### REFERENCE TO RELATED APPLICATIONS

This non-provisional patent application claims priority under 35 U.S.C. § 119(e) to provisional application U.S. Ser. No. 60/550,790 filed on Mar. 5, 2004 which is herein incorporated by reference in its entirety.

### FIELD OF THE INVENTION

The invention relates generally to the field of nucleic acid therapeutics and more particularly to methods of modulating the pharmacokinetics and biodistribution of aptamer therapeutics. The invention further relates to materials and methods for effecting the modulation of pharmacokinetics and biodistribution of novel aptamer compositions of the present invention.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (“mAbs”), are capable of specifically binding to selected targets and modulating the target's activity, e.g. through binding, aptamers may block their target's ability to function. Created by an in vitro selection process from pools of random sequence oligonucleotides (FIG. 1), aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarities, hydrophobic contacts, steric exclusion) that drive affinity and specificity in antibody-antigen complexes.

Aptamers have a number of desirable characteristics for use as therapeutics (and diagnostics) including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial leads, including therapeutic leads. In vitro selection allows the specificity and affinity of the aptamer to be tightly controlled and allows the generation of leads, including leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC. and the immune response is generally trained not to recognize nucleic acid fragments).

3) Administration. Whereas all currently approved antibody therapeutics are administered by intravenous infusion (typically over 2-4 hours), aptamers can also be administered by subcutaneous injection. This difference is primarily due to the comparatively low solubility and thus large volumes necessary for most therapeutic mAbs. With good solubility (>150 mg/mL) and comparatively low molecular weight (aptamer: 10-50 kDa; antibody: 150 kDa), a weekly dose of aptamer may be delivered by injection in a volume of less than 0.5 ml. In addition, the small size of aptamers allows them to penetrate into areas of conformational constrictions that do not allow antibodies or antibody fragments to penetrate, presenting yet another advantage of aptamer-based therapeutics or prophylaxis.

4) Scalability and cost. Therapeutic aptamers are chemically synthesized and consequently can be readily scaled as needed to meet production demand. Whereas difficulties in scaling production are currently limiting the availability of some biologics and the capital cost of a large-scale protein production plant is enormous, a single large-scale oligonucleotide synthesizer can produce upwards of 100 kg per year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to factors such as heat and denaturants and can be stored for extended periods (>1 yr) at room temperature as lyophilized powders

It would be beneficial to have materials and methods to modulate the pharmacodynamic and biodistribution properties of aptamer therapeutics to enhance their safety and efficacy as therapeutic agents.

### BRIEF DESCRIPTION OF THE DRAWINGS

FIG. 1 is a schematic representation of the in vitro aptamer selection (SELEX™) process using pools of random sequence oligonucleotides.

FIG. 2 is an illustration depicting the nucleotide sequence, composition, and secondary structure of the ARC83 aptamer (SEQ ID NO 5), in which R can be a 40 kDa PEG moiety, a 30 kDa PEG moiety, a 20 kDa PEG moiety, cholesterol, a Tat peptide, an Ant peptide, or an Arg₇ peptide. Positions of 2′-O-Me substitution are denoted by lowercase m and positions of 2′-F substitutions are denoted by lowercase f.

FIG. 3A is an illustration depicting a hybridization-based, dual-capture assay used for quantitative analysis of full-length aptamers in biological samples.

FIG. 3B is a graph depicting the quantitative analysis of various aptamer conjugates in rat plasma.

FIG. 4A is a graph depicting the plasma pharmacokinetic profiles of the ARC83, ARC120, ARC122 and ARC159 aptamer conjugates.

FIG. 4B is a graph depicting the plasma pharmacokinetic profiles of the ARC155, ARC156, ARC157 and ARC158 aptamer conjugates in rat plasma.

FIG. 5 is a graph depicting the biodistribution of the ARC83, ARC120, ARC122, ARC 155, ARC156, ARC157, ARC158 and ARC159 aptamers in rat tissues and organs.

FIG. 6A is a graph depicting the biodistribution of the [³H]-labeled ARC83 aptamer conjugates (expressed as percentage of dose administered) in rat organ or tissue samples harvested at 3, 12, or 24 hrs post-administration of the aptamer.

FIG. 6B is a graph depicting the biodistribution of the [³H]-labeled ARC120 aptamer conjugate in rat organ or tissue samples harvested at 3, 12, or 24 hrs post-administration of the aptamer.

FIG. 6C is a graph depicting the biodistribution of the [³H]-labeled ARC158 aptamer conjugate in rat organ or tissue samples harvested at 3, 12, or 24 hrs post-aptamer administration.

FIG. 6D is a graph depicting the biodistribution of the [³H]-labeled ARC 159 aptamer conjugate in rat organ or tissue samples harvested at 3, 12, or 24 hrs post-aptamer administration.

FIG. 7A is a graph depicting the biodistribution of the [³H]-labeled ARC83 aptamer conjugate (expressed as μg of [³H] label/g of tissue sample) in rat organ and tissue samples harvested at 3, 12, or 24 hrs post-aptamer administration.

FIG. 7B is a graph depicting the biodistribution of the [³H]-labeled ARC120 aptamer conjugate (expressed as μg of [³H] label/g of tissue sample) in rat organ or tissue samples harvested at 3, 12, or 24 hrs post-aptamer administration.

FIG. 7C is a graph depicting the biodistribution of the [³H]-labeled ARC 158 aptamer conjugate (expressed as μg of [³H] label/g of tissue sample) in rat organ or tissue samples harvested at 3, 12, or 24 hrs post-aptamer administration. FIG. 7D is a graph depicting the biodistribution of the [³H]-labeled ARC159 aptamer conjugate (expressed as μg of [³H] label/g of tissue sample) in rat organ or tissue samples harvested at 3, 12, or 24 hrs post-aptamer administration.

FIG. 8 is a graph depicting urinary elimination of various [³H]-labeled aptamer conjugates, ARC83, ARCH120, ARC158 and ARC159, in rats.

FIG. 9A is a graph depicting detection of the full-length ARC159 aptamer in rat urine using capillary gel electrophoresis.

FIG. 9B is a graph depicting detection of the full-length ARC 159 aptamer in rat urine using matrix-assisted laser desorption ionization-time of flight mass spectrometry (MALDI-TOF). The peaks corresponding to the ARC 159 aptamer and to a 20-mer internal standard are indicated in FIGS. 9A and 9B.

FIGS. 10A-10D are a series of images depicting quantitative whole-body autoradiography in inflammation-induced or non-inflamed mice dosed with radiolabeled aptamers. FIG. 10A is an image produced 3 hrs following intravenous (i.v.) administration of the [³H]-labeled ARC83 aptamer in a non-inflamed animal. FIG. 10B is an image produced 3 hrs post-i.v. administration of the [³H]-labeled ARC120 aptamer in an inflamed animal treated with carrageenan to induce inflammation locally in the right hind limb. FIG. 10C is an image produced 3 hrs post-i.v. administration of the [³H]-labeled ARC159 aptamer in an inflamed animal treated with carrageenan. FIG. 10D is an image produced 3 hrs post-i.v. administration of the [³H]-labeled ARC83 aptamer in an inflamed animal treated with carrageenan. FIG. 10E is a graph depicting the quantitative levels of various [³H]-aptamer equivalents (expressed in μg/g) in selected murine tissues. FIG. 10F is a table showing the level of uptake of radiolabeled aptamers into selected tissues (expressed as μg/g), as determined by whole-body autoradiography in which * denotes results for the control animal in which inflammation was not induced.

FIG. 11A is a graph depicting the levels of uptake of radiolabled aptamers in selected murine tissues (expressed as % of administered dose), as determined by whole body radiography. FIG. 11B is a table showing the level of uptake of radiolabeled aptamers into selected tissues (expressed as % of administered dose), as determined by whole-body autoradiography in which * denotes results for the control animal in which inflammation was not induced.

FIGS. 12A and 12B are images of quantitative autoradiography analysis of radiolabeled aptamer distribution to inflamed and non-inflamed hind limbs. FIG. 12A shows the distribution of the [³H]-labeled ARC83 aptamer in inflamed (right hind limb) or non-inflamed (left hind limb) tissues 3 hours after aptamer administration. FIG. 12B shows the distribution of the [³H]-ARC120 aptamer in inflamed (right hind limb) or non-inflamed (left hind limb) tissues 3 hrs after aptamer administration.

FIGS. 13A and 13B are graphs depicting the levels of [³H]-aptamer equivalents in inflamed and non-inflamed hind limb tissues. FIG. 13A depicts the concentrations of [³H]-aptamer equivalents in non-inflamed tissues (expressed in nM), and FIG. 13B depicts the relative concentrations of [³H]-aptamer equivalents as a ratio (R) of inflamed and non-inflamed tissues of the same animal.

FIGS. 14A-14C are a series of images depicting the cellular distribution of radiolabeled aptamers in murine kidney samples. FIG. 14A depicts the cellular distribution of the [³H]-labeled ARC83 aptamer; FIG. 14B depicts the cellular distribution of the [³H]-ARC120 aptamer; and FIG. 14C shows the cellular distribution of the [³H]-ARC159 aptamer.

FIGS. 15A-15C are a series of images depicting the cellular distribution of radiolabeled aptamers in murine liver samples. FIG. 15A shows the cellular distribution of the [³H]-labeled ARC83 aptamer; FIG. 15B depicts the cellular distribution of the [³H]-ARC120 aptamer; and FIG. 15C shows the cellular distribution of the [³H]-ARC159 aptamer.

FIG. 16 shows a schematic of the various PEGylation strategies representing standard mono-PEGylation, multiple PEGylation, and dimerization PEGylation.

### SUMMARY OF THE INVENTION

The present invention provides materials and methods to modulate the in vivo plasma pharmacokinetics and tissue distribution of aptamer therapeutics. In one embodiment, the present invention provides several aptamer compositions including those with modified nucleotides, for example, fully 2′-O-methylated oligonucleotides, and aptamer conjugates, whereby the aptamer is covalently coupled to a modifying moiety for which plasma pharmacokinetics and tissue distribution have been determined, such as, e.g., a high-molecular weight polyethylene glycol (PEG) polymer, a cell-permeating peptide, or a lipophilic molecule, such as cholesterol.

In one embodiment, the present invention provides methods to generate conjugates of the aptamers of the present invention with various modifying moieties that impart desired pharmacokinetic and biodistribution properties to the conjugated aptamer therapeutics. These modifying moieties include, without limitation, high or low molecular weight polyethylene glycol (PEG) polymers, cell-permeating peptides, or lipophilic molecules, such as cholesterol.

In one embodiment, the aptamer compositions of the present invention prepared according to the methods of the present invention exhibit a wide range of mean residence times in circulation (0.6-16 hr) and significant variation in distribution levels among different organs and tissues.

In some embodiments, conjugation of an aptamer with a PEG polymer comprising a molecular weight of no more than 20 kDa, no more than 10 kDa or no more than 5 kDa, preferably of about 20 kDa, prolongs aptamer circulatory half-life and enhances exposure to tissues, while reducing both the extent of aptamer distribution to the kidneys and the rate of urinary elimination. In another embodiment, a non-conjugated, fully 2′-O-Me aptamer composition shows rapid clearance from circulation, and elimination with intact aptamer being detectable in urine at 48 hr post-administration.

In one embodiment of the present invention, the modulation of pharmacokinetic and biodistribution properties of aptamers of the present invention enhance the safety and efficacy of aptamers as therapeutic agents.

The present invention provides materials and methods to modulate the effects of conjugation of small molecule, peptide, or polymer internal or terminal groups on the pharmacokinetics and biodistribution of stabilized aptamer compositions in vivo.

In one embodiment, the present invention provides materials and methods to determine the levels of aptamer conjugates in biological samples by radiometric quantification and by a hybridization-based dual probe capture assay with an enzyme-linked fluorescent readout.

In one embodiment of the present invention, conjugation with a modifying moiety and/or changing the chemical composition of the nucleotides alters fundamental aspects of aptamer residence in circulation and distribution to tissues.

Another embodiment the present invention provides a method of targeting biodistribution of an aptamer conjugate of the present invention to a particular organ or tissue by the selection of a modifying moiety that increases distribution of the complex to that targeted tissue.

In one embodiment, the present invention provides an aptamer therapeutic with improved biodistribution to well-perfused tissues or organs.

In one embodiment, the present invention provides an aptamer therapeutic conjugate with improved biodistribution to inflamed tissues or organs.

In one aspect, the present invention provides a method of modulating the pharmacokinetic and biodistribution properties of an aptamer therapeutic by providing a reactive aptamer therapeutic having at least one reactive moiety, wherein the reactive moiety is reacted with a modifying moiety selected from the group consisting of a polyethylene glycol (PEG) polymer, a cell-permeating peptide, cholesterol, an affinity tag, an affinity label, and a diagnostic imaging label; providing a modified aptamer therapeutic having at least one modified nucleic acid residue incorporated therein; or providing a modified, reactive aptamer therapeutic having at least one modified nucleic acid residue incorporated therein, and also having at least one reactive moiety, wherein the reactive moiety is reacted with a modifying moiety selected from the group consisting of a polyethylene glycol (PEG) polymer, a cell-permeating peptide, cholesterol, an affinity tag, and an affinity label. In one embodiment, the aptamer therapeutic has at least one reactive moiety located at least one terminus. In another embodiment, the aptamer therapeutic has at least one internal-reactive moiety. In one embodiment, the aptamer therapeutic has at least one modified nucleic acid residue having a 2′-sugar modification. In another embodiment, the aptamer therapeutic has at least one modified nucleic acid residue having a backbone modification.

The PEG polymers used in this method have a molecular weight of about 10 kDa, and in some embodiments, the PEG polymers have a molecular weight of 20 kDa, 30 kDa, 40 or 60 kDa.

In another aspect, the present invention provides a method of targeting an aptamer therapeutic to a specific tissue or organ by delivering a reactive aptamer therapeutic to a subject, wherein the reactive aptamer therapeutic includes at least one reactive moiety that is reacted with a modifying moiety selected from the group consisting of a polyethylene glycol (PEG) polymer, a cell-permeating peptide, cholesterol, an affinity tag, an affinity label, and a diagnostic imaging label; delivering a modified aptamer therapeutic to a subject, wherein the modified aptamer therapeutic includes at least one modified nucleic acid residue incorporated therein; or delivering a modified, reactive aptamer therapeutic to a subject, wherein the modified, reactive aptamer therapeutic has at least one modified nucleic acid residue incorporated therein, and also has at least one reactive moiety, wherein the reactive moiety is reacted with a modifying moiety selected from the group consisting of a polyethylene glycol (PEG) polymer, a cell-permeating peptide, cholesterol, an affinity tag, and an affinity label. In one embodiment, the specific tissue or organ is a well-perfused tissue or organ. The well-perfused tissue is, for example, selected from inflamed tissue, bone marrow, liver, lung, myocardium, spleen, and kidney. In another embodiment, the specific tissue or organ for aptamer delivery is mediastinal lymph nodes. In another embodiment, the specific tissue for aptamer delivery is selected from the group consisting of: solid tumor, and cancerous tissue or cancerous growth.

In another aspect, the present invention provides a method of treating an inflammatory disorder by administering a reactive aptamer therapeutic to a subject, wherein the reactive aptamer therapeutic includes at least one reactive moiety that is reacted with a modifying moiety selected from the group consisting of a polyethylene glycol (PEG) polymer, a cell-permeating peptide, cholesterol, an affinity tag, and an affinity label; administering a modified aptamer therapeutic to a subject, wherein the modified aptamer therapeutic includes at least one modified nucleic acid residue incorporated therein to a subject; or administering a modified, reactive aptamer therapeutic to a subject, wherein modified, reactive aptamer therapeutic includes at least one modified nucleic acid residue incorporated therein, and also includes at least one reactive moiety, wherein the reactive moiety is reacted with a modifying moiety selected from the group consisting of a polyethylene glycol (PEG) polymer, a cell-permeating peptide, cholesterol, an affinity tag, and an affinity label.

In one embodiment, the aptamer therapeutic has at least one reactive moiety located at least one terminus. In another embodiment, the aptamer therapeutic has at least one internal reactive moiety. In one embodiment, the aptamer therapeutic has at least one modified nucleic acid residue having a 2′-sugar modification. In another embodiment, the aptamer therapeutic has at least one modified nucleic acid residue having a backbone modification.

The PEG polymers used in this method have a molecular weight of about 10 kDa, and in some embodiments, the PEG polymers have a molecular weight of 20 kDa, 30 kDa, 40 or 60 kDa.

The invention also provides therapeutic aptamer conjugate compositions that include a nucleic acid moiety conjugated to a polyethylene glycol (PEG) polymer. The PEG polymers used in these compositions have a molecular weight of at least 10 kDa, and in some embodiments, the PEG polymers have a molecular weight of 20 kDa, 30 kDa or 40 kDa. In other embodiments of this aspect of the invention, the PEG polymers used in these compositions have a molecular weight or no more than 20 kDa, 10 kDa or 5 kDa.

In another aspect, the invention provides therapeutic aptamer compositions that include a nucleic acid moiety conjugated to a peptide. In one embodiment, the peptide is a cell permeating peptide. For example, in one embodiment, the cell permeating peptide is HIV-Tat peptide. In another embodiment, the cell-permeating peptide is Antennapedia (Ant)-derived peptide. In yet another embodiment, the cell-permeating peptide is poly-Arg7.

In another aspect, the invention provides therapeutic aptamer compositions that include a nucleic acid moiety conjugated to a small molecule. In one embodiment, the small molecule is selected from the group consisting of affinity tags, cell-permeable moieties and affinity labels. In another embodiment, the cell-permeable moiety is a lipophilic molecule. For example, in one embodiment, the lipophilic molecule is cholesterol.

In another aspect, the invention provides therapeutic aptamer compositions that include a nucleic acid moiety having at least one altered nucleic acid residue, wherein the alteration is selected from the group consisting of a 2′-sugar modification and a backbone modification. In a particular embodiment, the invention provides an aptamer comprising a nucleic acid sequence having at least one altered nucleic acid residue conjugated to therapeutic molecule, such as a cytotoxin, useful for the treatment of cancer.

In another aspect of the invention, a method of modulating in vivo aptamer distribution, comprising administering an aptamer composition to a subject wherein the chemical composition of the aptamer nucleic acid sequence is formulated to modulate a pre-selected aptamer distribution property in vivo, and wherein, the pre-selected aptamer distribution property to be modulated is not reduction of the aptamer plasma clearance rate due to reduction in aptamer enzymatic degradation, is provided. In some embodiments of this aspect of the invention, the pre-selected aptamer distribution property is preferential accumulation of a first aptamer in a predetermined tissue or organ. In some embodiments the predetermined tissue or organ is selected from the group consisting of kidney and gastrointestinal tract. In some embodiments of this aspect of the invention, the predetermined aptamer distribution property is the rate of aptamer elimination from the body, e.g. via the urine. In some aspects of this embodiment, the aptamer elimination rate is increased relative to a second aptamer having a chemical composition different than that of the nucleic acid sequence of the first aptamer, preferably relative to a second aptamer having the same nucleic acid sequence but a different chemical composition relative to the first aptamer, and in some embodiments the second aptamer comprises all deoxy nucleotides.

In another embodiment of this aspect of the invention a method for treating, preventing and/or ameliorating a disease or disorder selected from the group consisting of consisting of cancer and acute care conditions, comprising administering the first aptamer to a subject, e.g. a human subject, is provided. In some embodiments, e.g. where the disease to be treated is cancer, the nucleic acid sequence of the first aptamer is conjugated to a cytotoxin. In some embodiments the aptamer composition comprising the first aptamer is administered orally.

In some embodiments of this aspect of the invention, the first aptamer comprises at least one nucleic acid residue having a 2′-sugar modification, e.g. a 2′-OMe substitution. In some embodiments, the first aptamer comprises more than forty percent 2′-OMe substituted nucleotides, in some embodiments more than fifty percent, in some embodiments more than sixty percent; in some embodiments more than 75 percent, in some embodiments more than ninety percent and in some embodiments all of the aptamer nucleotides are 2′-OMe substituted.

In another aspect of the invention, a method of modulating in vivo aptamer distribution, comprising administering an aptamer composition to a subject wherein the aptamer composition is formulated to reduce aptamer clearance rate due to renal filtration and wherein the aptamer is conjugated to a polyethylene glycol moiety of not more than 20 kDa, not more than 10 kDa or not more than 5 kDa is provided. In some embodiments of this aspect the polyethylene glycol moiety has molecular weight of less than 20 kDa, 10 kDa or 5 kDa.

In yet another aspect of the invention, a method of modulating in vivo aptamer distribution, comprising administering an aptamer composition to a subject wherein the chemical composition of the aptamer is formulated to modulate a pre-selected aptamer distribution property in vivo, and wherein, the pre-selected aptamer distribution property to be modulated is not reduction of aptamer clearance rate due to renal filtration is provided. In some embodiments of this aspect of the invention the aptamer is conjugated to a polyethylene glycol moiety. In some embodiments the polyethylene glycol moiety comprises a molecular weight selected from the group consisting of: 10, 20, 30, 40 and 60 kDa. In other embodiments, the polyethylene glycol moiety comprises a molecular weight of no more than 20, 10, or 5 kDa. In other embodiments of this aspect of the invention the polyethylene glycol moiety comprises a molecular weight of less than 20, 10, or 5 kDa.

In some embodiments of this aspect of the invention, the pre-selected aptamer distribution property is preferential accumulation in a predetermined tissue or organ. In some embodiments, the predetermined tissue or organ is a highly perfused tissue or organ. In some embodiments, the highly perfused tissue or organ is selected from the group consisting of liver, spleen, heart, lung and mediastinal lymph node. In some embodiments the predetermined tissue or organ is selected from the group consisting of: inflamed tissue, tumor tissue, and cancerous tissue.

In some embodiments of this aspect of the invention, the aptamer binds specifically to a target that mediates allergic disease, inflammatory disease, rheumatoid arthritis, psoriasis or asthma. In other embodiments of this aspect of the invention, the aptamer binds specifically to a target that mediates allergic disease, inflammatory disease, rheumatoid arthritis, psoriasis or asthma and the method further comprising administering the aptamer composition to a subject to treat or prevent allergic disease, inflammatory disease, rheumatoid arthritis, psoriasis or asthma respectively. In some embodiments of this aspect of the invention the subject is a vertebrate, preferably a mammal, more preferably a human. In some embodiments of this aspect of the invention the aptamer composition is administered systemically.

In some embodiments of this aspect of the invention, the aptamer binds specifically to a target that mediates cancer or infectious disease. In some embodiments of this aspect of the invention wherein the aptamer binds specifically to a target that mediates cancer or infectious disease a method for treating disease comprising administering the aptamer composition to a subject to treat or prevent cancer or infectious disease respectively is provided.

In another embodiment of this aspect of the invention a method for treating a disease or disorder of a highly perfused tissue or organ comprising administering a PEGylated aptamer composition to a subject is provided. In still another embodiment of this aspect of the invention a method of treating or preventing cancer comprising administering PEGylated aptamer composition to a subject is provided.

In an embodiment of another aspect of this invention, the aptamer is conjugated to a peptide, particularly and Ant or Tat peptide. In some embodiments, the peptide conjugated aptamer preferentially accumulated in the kidney or kidney tissue.

In an embodiment of another aspect of the invention, the pre-selected aptamer distribution property to be modulated the rate of conjugated-aptamer clearance from the plasma. In some embodiments the plasma aptamer clearance rate is increased. In some embodiments the plasma aptamer clearance rate is increased where the aptamer is conjugated to a peptide, particularly Tat, or to cholesterol.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

The SELEX™ Method

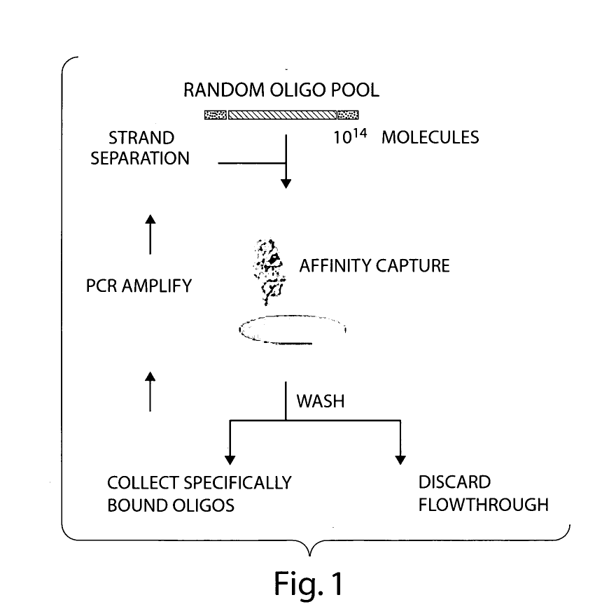

A suitable method for generating an aptamer is with the process entitled “Systematic Evolution of Ligands by Exponential Enrichment” (“SELEX™”) generally depicted in FIG. 1. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX™-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotides comprising randomized sequences derived from chemical synthesis on a standard DNA synthesizer. The oligonucleotides can be modified or unmodified DNA, RNA or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a preselected -purpose such as, CpG motifs described further below, hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g., U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 10¹⁴-10¹⁶ individual molecules, a number sufficient for most SELEX™ experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be for example, RNA, DNA or RNA/DNA hybrids. In those instances where an RNA library is to be used as the starting library it is typically generated by transcribing a DNA library in vitro using T7 RNA polymerase or modified T7 RNA polymerases and purified. The nucleic acid library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 10¹⁴ different nucleic acid species but may be used to sample as many as about 10¹⁸ different nucleic acid species. Generally, nucleic acid aptamer molecules are selected in a 5 to 20 cycle procedure. In one embodiment, heterogeneity is introduced only in the initial selection stages and does not occur throughout the replicating process.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20 to about 50 nucleotides and in some embodiments, about 30 to about 40 nucleotides. In one example, the 5′-fixed:random:3′-fixed sequence comprises a random sequence of about 30 to about 50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photo reactive groups capable of binding and/or photo-crosslinking to and/or photo-inactivating a target molecule. U.S. Pat. No. 5,567,588 and U.S. Pat. No. 5,861,254 describe SELEX™ based methods which achieve highly efficient partitioning between oligonucleotides having high and low affinity for a target molecule. U.S. Pat. No. 5,496,938 describes methods for obtaining improved nucleic acid ligands after the SELEX process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules such as nucleic acid-binding proteins and proteins not known to bind nucleic acids as part of their biological function as well as cofactors and other small molecules. For example, U.S. Pat. No. 5,580,737 discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; (e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX™, cycles of selection and amplification are repeated as necessary until a desired goal is achieved.

One potential problem encountered in the use of nucleic acids as therapeutics and vaccines is that oligonucleotides in their phosphodiester form may be quickly degraded in body fluids by intracellular and extracellular enzymes such as endonucleases and exonucleases before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX™-identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, and unusual base-pairing combinations such as the isobases isocytidine and isoguanosine. Modifications can also include 3′ and 5′ modifications such as capping. In some embodiments of the instant invention, the nucleic acid ligands are RNA molecules that are 2′-OMe modified on the sugar moiety of at least one nucleotide, preferably on at least 25% of the nucleotides, preferably on at least 40% of the nucleotides, preferably on at least 50% of the nucleotides, preferably on at least 60% of the nucleotides, preferably on at least 75% of the nucleotides, preferably on at least 90% of the nucleotides and preferably on all of the nucleotides.

In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atom.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX™ process modifications or post-SELEX™ process modifications (modification of previously identified unmodified ligands) or may be made by incorporation into the SELEX™ process.

Pre-SELEX™ process modifications or those made by incorporation into the SELEX™ process yield nucleic acid ligands with both specificity for their SELEX™ target, and in some embodiments improved stability, e.g., in vivo stability. Post-SELEX™ process modifications made to nucleic acid ligands may also result in improved stability, e.g., in vivo stability without adversely affecting the binding capacity of the nucleic acid ligand.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

The identification of nucleic acid ligands to small, flexible peptides via the SELEX™ method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

The aptamers of the present invention with specificity and binding affinity to a given target(s) of the present invention are typically selected by the SELEX™ process as described herein. As part of the SELEX™ process, the sequences selected to bind to the target are then optionally minimized to determine the minimal sequence having the desired binding affinity. The selected sequences and/or the minimized sequences are optionally optimized by performing random or directed mutagenesis of the sequence, for example, to increase binding affinity, to determine which positions in the sequence are essential for binding activity, to stabilize the aptamer molecules against degradation in vivo, to affect in vivo distribution, e.g., biodistribution and/or pharmacokinetics, particularly plasma pharmacokinetics. Additionally, selections can be performed with sequences incorporating modified nucleotides, e.g. to increase binding affinity, to determine which positions in the sequence are essential for binding activity, to stabilize the aptamer molecules against degradation in vivo, to affect in vivo distribution, for example, biodistribution and/or pharmacokinetic, particularly plasma pharmacokinetics.

2′ Modified SELEX™

In order for an aptamer to be suitable for use as a therapeutic, it is preferably inexpensive to synthesize, safe and stable in vivo. Wild-type RNA and DNA aptamers are typically not sufficiently stable in vivo because of their susceptibility to degradation by nucleases. Resistance to nuclease degradation can be greatly increased by the incorporation of modifying groups at the 2′-position.

Fluoro and amino groups have been successfully incorporated into oligonucleotide pools from which aptamers have been subsequently selected. However, these modifications greatly increase the cost of synthesis of the resultant aptamer, and may introduce safety concerns in some cases because of the possibility that the modified nucleotides could be recycled into host DNA by degradation of the modified oligonucleotides and subsequent use of the nucleotides as substrates for DNA synthesis.

Aptamers that contain 2′-O-methyl (“2′-OMe”) nucleotides, as provided herein, overcome many of these drawbacks. Oligonucleotides containing 2′-OMe nucleotides are nuclease-resistant and inexpensive to synthesize. Although 2′-OMe nucleotides are ubiquitous in biological systems, natural polymerases do not accept 2′-OMe NTPs as substrates under physiological conditions, thus there are no safety concerns over the recycling of 2′-OMe nucleotides into host DNA.

Additionally, in one aspect of the present invention it has been discovered that by specifically formulating the composition of an aptamer nucleic acid sequence, e.g. modifying a percentage of the nucleotides to comprise 2′-OMe, the pharmacokinetics and/or biodistribution of aptamer may be tuned to achieve a predetermined goal. In some embodiments, more than 25% of the nucleotides of the aptamer sequence are 2′-OMe, in some embodiments more than 40% of the nucleotides are 2′-OMe, in some embodiments more than 50% of the nucleotide are 2′-Ome, in some embodiments more than 60% of the nucleotides are 2′-OMe, in some embodiments more than 75% of the nucleotides are 2′-OMe, in some embodiments more than 90% of the nucleotides are 2′-OMe and in some embodiments all the nucleotides are 2′-OMe. In some embodiments, the plasma clearance rate of a 2′-OMe containing aptamer is increased relative to an aptamer comprising fewer or no 2′-OMe substitutions. In some embodiments, the 2′-OMe containing aptamer preferentially accumulates in the kidney or gastrointestinal tract.

The SELEX™ method used to generate 2′-modified aptamers is described, e.g., in U.S. Provisional Patent Application Ser. No. 60/430,761, filed Dec. 3, 2002, U.S. Provisional Patent Application Ser. No. 60/487,474, filed Jul. 15, 2003, U.S. Provisional Patent Application Ser. No. 60/517,039, filed Nov. 4, 2003, U.S. patent application Ser. No. 10/729,581, filed Dec. 3, 2003, and U.S. Patent Application No. 10/873,856, filed Jun. 21, 2004, entitled “Method for in vitro Selection of 2′-O-methyl Substituted Nucleic Acids”, each of which is herein incorporated by reference in its entirety.

The present invention includes aptamers that bind to and modulate the function of a target which contain modified nucleotides (e.g., nucleotides which have a modification at the 2′ position) to make the oligonucleotide more stable than the unmodified oligonucleotide to enzymatic, chemical, thermal, physical degradation as well as to modulate the pharmacokinetics and/or biodistribution of the modified aptamer in a subject.

Although there are several examples of 2′-OMe containing aptamers in the literature (see, e.g., Green et al., Current Biology 2, 683-695, 1995) these were generated by the in vitro selection of libraries of modified transcripts in which the C and U residues were 2′-fluoro (2′-F) substituted and the A and G residues were 2′-OH. Once functional sequences were identified then each A and G residue was tested for tolerance to 2′-OMe substitution, and the aptamer was re-synthesized having all A and G residues which tolerated 2′-OMe substitution as 2′-OMe residues. Most of the A and G residues of aptamers generated in this two-step fashion tolerate substitution with 2′-OMe residues, although, on average, approximately 20% do not. Consequently, aptamers generated using this method tend to contain from two to four 2′-OH residues, and stability and cost of synthesis are compromised as a result. By incorporating modified nucleotides into the transcription reaction which generate stabilized oligonucleotides used in oligonucleotide pools from which aptamers are selected and enriched by SELEX™ (and/or any of its variations and improvements, including those described herein), the methods of the present invention eliminate the need for stabilizing the selected aptamer oligonucleotides (e.g., by resynthesizing the aptamer oligonucleotides with modified nucleotides).

In one embodiment, the present invention provides aptamers comprising combinations of 2′-OH, 2′-F, 2′-deoxy, and/or 2′-OMe modifications of the ATP, GTP, CTP, TTP, and UTP nucleotides. In another embodiment, the present invention provides aptamers comprising combinations of 2′-OH, 2′-F, 2′-deoxy, 2′-OMe, 2′-NH₂, and/or 2′-methoxyethyl modifications of the ATP, GTP, CTP, TTP, and UTP nucleotides. In another embodiment, the present invention provides aptamers comprising combinations of 2′-OH, 2′-F, 2′-deoxy, 2′-OMe, 2′-NH₂, and 2′-methoxyethyl modifications of the ATP, GTP, CTP, TTP, and UTP nucleotides. In another embodiment, the present invention provides aptamers comprising combinations of 2′-OH, 2′-deoxy and/or 2′-OMe ATP, GTP, CTP, TTP and/or UTP nucleotides. In a particular embodiment, the present invention provides aptamers comprising all 2′-OMe ATP, GTP, CTP, TTP and/or UTP nucleotides.

2′ modified aptamers of the invention may be created using modified polymerases, e.g., a modified T7 polymerase, having a rate of incorporation of modified nucleotides having bulky substituents at the furanose 2′ position that is higher than that of wild-type polymerases. For example, a mutant T7 polymerase (Y639F) in which the tyrosine residue at position 639 has been changed to phenylalanine readily utilizes 2′deoxy, 2′amino-, and 2′fluoro-nucleotide triphosphates (NTPs) as substrates and has been widely used to synthesize modified RNAs for a variety of applications. However, this mutant T7 polymerase reportedly can not readily utilize (i.e., incorporate) NTPs with bulky 2′-substituents such as 2′-OMe or 2′-azido (2′-N₃) substituents. For incorporation of bulky 2′ substituents, a T7 polymerase mutant (Y639F/H784A) having the histidine at position 784 changed to an alanine residue in addition to the Y639F mutation has been described and has been used in limited circumstances to incorporate modified pyrimidine NTPs. See Padilla, R. and Sousa, R., Nucleic Acids Res., 2002, 30(24): 138. A mutant T7 polymerase (H784A) having the histidine at position 784 changed to an alanine residue has also been described. Padilla et al., Nucleic Acids Research, 2002, 30: 138. In both the Y639F/H784A mutant and H784A mutant T7 polymerases, the change to a smaller amino acid residue such as alanine allows for the incorporation of bulkier nucleotide substrates, e.g., 2′-OMe substituted nucleotides.

Generally, it has been found that under the conditions disclosed herein, the Y693F mutant can be used for the incorporation of all 2′-OMe substituted NTPs except GTP and the Y639F/H784A mutant can be used for the incorporation of all 2′-OMe substituted NTPs including GTP. It is expected that the H784A mutant possesses properties similar to the Y639F and the Y639F/H784A mutants when used under the conditions disclosed herein.

2′-modified oligonucleotides may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this way, transcripts, or pools of transcripts are generated using any combination of modifications, including for example, ribonucleotides (2′-OH), deoxyribonucleotides (2′-deoxy), 2′-F, and 2′-OMe nucleotides. A transcription mixture containing 2′-OMe C and U and 2′-OH A and G is referred to as an “rRmY” mixture and aptamers selected therefrom are referred to as “rRmY” aptamers. A transcription mixture containing deoxy A and G and 2′-OMe U and C is referred to as a “dRmY” mixture and aptamers selected therefrom are referred to as “dRmY” aptamers. A transcription mixture containing 2′-OMe A, C, and U, and 2′-OH G is referred to as a “rGmH” mixture and aptamers selected therefrom are referred to as “rGmH” aptamers. A transcription mixture alternately containing 2′-OMe A, C, U and G and 2′-OMe A, U and C and 2′-F G is referred to as an “alternating mixture” and aptamers selected therefrom are referred to as “alternating mixture” aptamers. A transcription mixture containing 2′-OMe A, U, C, and G, where up to 10% of the G's are ribonucleotides is referred to as a “r/mGmH” mixture and aptamers selected therefrom are referred to as “r/mGmH” aptamers. A transcription mixture containing 2′-OMe A, U, and C, and 2′-F G is referred to as a “fGmH” mixture and aptamers selected therefrom are referred to as “fGmH” aptamers. A transcription mixture containing 2′-OMe A, U, and C, and deoxy G is referred to as a “dGmH” mixture and aptamers selected therefrom are referred to as “dGmH” aptamers. A transcription mixture containing deoxy A, and 2′-OMe C, G and U is referred to as a “dAmB” mixture and aptamers selected therefrom are referred to as “dAmB” aptamers, and a transcription mixture containing all 2′-OH nucleotides is referred to as a “rN” mixture and aptamers selected therefrom are referred to as “rN” or “rRrY” aptamers. A “mRmY” aptamer is one containing all 2′-O-methyl nucleotides and is usually derived from a r/mGmH oligonucleotide by post-SELEX™ replacement, when possible, of any 2′-OH Gs with 2′-OMe Gs.

A preferred embodiment includes any combination of 2′-OH, 2′-deoxy and 2′-OMe nucleotides. A more preferred embodiment includes any combination of 2′-deoxy and 2′-OMe nucleotides. An even more preferred embodiment is with any combination of 2′-deoxy and 2′-OMe nucleotides in which the pyrimidines are 2′-OMe (such as dRmY, mRmY or dGmH).

Incorporation of modified nucleotides into the aptamers of the invention is accomplished before (pre-) the selection process (e.g., a pre-SELEX™ process modification). Optionally, aptamers of the invention in which modified nucleotides have been incorporated by pre-SELEX™ process modification can be further modified by post-SELEX™ process modification (i.e., a post-SELEX™ process modification after a pre-SELEX™ modification). Pre-SELEX™ process modifications yield modified nucleic acid ligands with specificity for the SELEX™ target and also improved in vivo stability. Post-SELEX™ process modifications, i.e., modification (e.g., truncation, deletion, substitution or additional nucleotide modifications of previously identified ligands having nucleotides incorporated by pre-SELEX™ process modification) can result in a further improvement of in vivo stability without adversely affecting the binding capacity of the nucleic acid ligand having nucleotides incorporated by pre-SELEX™ process modification.

To generate pools of 2′-modified (e.g., 2′-OMe) RNA transcripts in conditions under which a polymerase accepts 2′-modified NTPs the preferred polymerase is the Y693F/H784A mutant or the Y693F mutant. Other polymerases, particularly those that exhibit a high tolerance for bulky 2′-substituents, may also be used in the present invention. Such polymerases can be screened for this capability by assaying their ability to incorporate modified nucleotides under the transcription conditions disclosed herein.

A number of factors have been determined to be important for the transcription conditions useful in the methods disclosed herein. For example, increases in the yields of modified transcript are observed when a leader sequence is incorporated into the 5′ end of a fixed sequence at the 5′ end of the DNA transcription template, such that at least about the first 6 residues of the resultant transcript are all purines.

Another important factor in obtaining transcripts incorporating modified nucleotides is the presence or concentration of 2′-OH GTP. Transcription can be divided into two phases: the first phase is initiation, during which an NTP is added to the 3′-hydroxyl end of GTP (or another substituted guanosine) to yield a dinucleotide which is then extended by about 10-12 nucleotides; the second phase is elongation, during which transcription proceeds beyond the addition of the first about 10-12 nucleotides. It has been found that small amounts of 2′-OH GTP added to a transcription mixture containing an excess of 2′-OMe GTP are sufficient to enable the polymerase to initiate transcription using 2′-OH GTP, but once transcription enters the elongation phase the reduced discrimination between 2′-OMe and 2′-OH GTP, and the excess of 2′-OMe GTP over 2′-OH GTP allows the incorporation of principally the 2′-OMe GTP.

Another important factor in the incorporation of 2′-OMe substituted nucleotides into transcripts is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions. To obtain the greatest yields of maximally 2′ substituted O-methylated transcripts (i.e., all A, C, and U and about 90% of G nucleotides), concentrations of approximately 5 mM magnesium chloride and 1.5 mM manganese chloride are preferred when each NTP is present at a concentration of 0.5 mM. When the concentration of each NTP is 1.0 mM, concentrations of approximately 6.5 mM magnesium chloride and 2.0 mM manganese chloride are preferred. When the concentration of each NTP is 2.0 mM, concentrations of approximately 9.6 mM magnesium chloride and 2.9 mM manganese chloride are preferred. In any case, departures from these concentrations of up to two-fold still give significant amounts of modified transcripts.

Priming transcription with GMP or guanosine is also important. This effect results from the specificity of the polymerase for the initiating nucleotide. As a result, the 5′-terminal nucleotide of any transcript generated in this fashion is likely to be 2′-OH G. The preferred concentration of GMP (or guanosine) is 0.5 mM and even more preferably 1 mM. It has also been found that including PEG, preferably PEG-8000, in the transcription reaction is useful to maximize incorporation of modified nucleotides.

For maximum incorporation of 2′-OMe ATP (100%), UTP (100%), CTP (100%) and GTP (˜90%) (“r/mGmH”) into transcripts the following conditions are preferred: HEPES buffer 200 mM, DTT 40 mM, spermidine 2 mM, PEG-8000 10% (w/v), Triton X-100 0.01% (w/v), MgCI₂ 5 mM (6.5 mM where the concentration of each 2′-OMe NTP is 1.0 mM), MnCI₂ 1.5 mM (2.0 mM where the concentration of each 2′-OMe NTP is 1.0 mM), 2′-OMe NTP (each) 500 μM (more preferably, 1.0 mM), 2′-OH GTP 30 μM, 2′-OH GMP 500 μM, pH 7.5, Y639F/H784A T7 RNA Polymerase 15 units/ml, inorganic pyrophosphatase 5 units/ml, and an all-purine leader sequence of at least 8 nucleotides long. As used herein, one unit of the Y639F/H784A mutant T7 RNA polymerase (or any other mutant T7 RNA polymerase specified herein) is defined as the amount of enzyme required to incorporate 1 nmole of 2′-OMe NTPs into transcripts under the r/mGmH conditions. As used herein, one unit of inorganic pyrophosphatase is defined as the amount of enzyme that will liberate 1.0 mole of inorganic orthophosphate per minute at pH 7.2 and 25° C.

For maximum incorporation (100%) of 2′-OMe ATP, UTP and CTP (“rGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F T7 RNA Polymerase 15 units/ml, inorganic pyrophosphatase 5 units/ml, and an all-purine leader sequence of at least 8 nucleotides long.

For maximum incorporation (100%) of 2′-OMe UTP and CTP (“rRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F/H784A T7 RNA Polymerase 15 units/ml, inorganic pyrophosphatase 5 units/ml, and an all-purine leader sequence of at least 8 nucleotides long.

For maximum incorporation (100%) of deoxy ATP and GTP and 2′-OMe UTP and CTP (“dRmY”) into transcripts the following conditions are preferred: HEPES buffer 200 mM, DTT 40 mM, spermine 2 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For maximum incorporation (100%) of 2′-OMe ATP, UTP and CTP and 2′-F GTP (“fGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For maximum incorporation (100%) of deoxy ATP and 2′-OMe UTP, GTP and CTP (“dAmB”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For each of the above (a) transcription is preferably performed at a temperature of from about 20° C. to about 50° C., preferably from about 30° C. to 45° C., and more preferably about 37° C. for a period of at least two hours and (b) 50-300 nM of a double stranded DNA transcription template is used (200 nM template is used in round 1 to increase diversity (300 nM template is used in dRmY transcriptions)), and for subsequent rounds approximately 50 nM, a 1/10 dilution of an optimized PCR reaction, using conditions described herein, is used). The preferred DNA transcription templates are described below (where ARC254 and ARC256 transcribe under all 2′-OMe conditions and ARC255 transcribes under rRmY conditions).

SEQ ID NO 1

ARC254
5′-CATCGATGCTAGTCGTAACGATCCNNNNNNNNNNNNNNNNNNNNNNN
NNNNNNNCGAGAACGTTCTCTCCTCTCCCTATAGTGAGTCGTATTA-3′

SEQ ID NO 2

ARC255
5′-CATGCATCGCGACTGACTAGCCGNNNNNNNNNNNNNNNNNNNNNNNN
NNNNNNGTAGAACGTTCTCTCCTCTCCCTATAGTGAGTCGTATTA-3′

SEQ ID NO 3

ARC256
5′-CATCGATCGATCGATCGACAGCGNNNNNNNNNNNNNNNNNNNNNNNN
NNNNNNGTAGAACGTTCTCTCCTCTCCCTATAGTGAGTCGTATTA-3′

Under rN transcription conditions of the present invention, the transcription reaction mixture comprises 2′-OH adenosine triphosphates (ATP), 2′-OH guanosine triphosphates (GTP), 2′-OH cytidine triphosphates (CTP), and 2′-OH uridine triphosphates (UTP). The modified oligonucleotides produced using the rN transcription mixtures of the present invention comprise substantially all 2′-OH adenosine, 2′-OH guanosine, 2′-OH cytidine, and 2′-OH uridine. In a preferred embodiment of rN transcription, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-OH cytidine, and at least 80% of all uridine nucleotides are 2′-OH uridine. In a more preferred embodiment of rN transcription, the resulting modified oligonucleotides of the present invention comprise a sequence where at least 90% of all adenosine nucleotides are 2′-OH adenosine, at least 90% of all guanosine nucleotides are 2′-OH guanosine, at least 90% of all cytidine nucleotides are 2′-OH cytidine, and at least 90% of all uridine nucleotides are 2′-OH uridine. In a most preferred embodiment of rN transcription, the modified oligonucleotides of the present invention comprise a sequence where 100% of all adenosine nucleotides are 2′-OH adenosine, 100% of all guanosine nucleotides are 2′-OH guanosine, 100% of all cytidine nucleotides are 2′-OH cytidine, and 100% of all uridine nucleotides are 2′-OH uridine.

Under rRmY transcription conditions of the present invention, the transcription reaction mixture comprises 2′-OH adenosine triphosphates, 2′-OH guanosine triphosphates, 2′-O-methyl cytidine triphosphates, and 2′-O-methyl uridine triphosphates. The modified oligonucleotides produced using the rRmY transcription mixtures of the present invention comprise substantially all 2′-OH adenosine, 2′-OH guanosine, 2′-O-methyl cytidine and 2′-O-methyl uridine. In a preferred embodiment, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In a more preferred embodiment, the resulting modified oligonucleotides comprise a sequence where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine In a most preferred embodiment, the resulting modified oligonucleotides comprise a sequence where 100% of all adenosine nucleotides are 2′-OH adenosine, 100% of all guanosine nucleotides are 2′-OH guanosine, 100% of all cytidine nucleotides are 2′-O-methyl cytidine and 100% of all uridine nucleotides are 2′-O-methyl uridine.

Under dRmY transcription conditions of the present invention, the transcription reaction mixture comprises 2′-deoxy adenosine triphosphates, 2′-deoxy guanosine triphosphates, 2′-O-methyl cytidine triphosphates, and 2′-O-methyl uridine triphosphates. The modified oligonucleotides produced using the dRmY transcription conditions of the present invention comprise substantially all 2′-deoxy adenosine, 2′-deoxy guanosine, 2′-O-methyl cytidine, and 2′-O-methyl uridine. In a preferred embodiment, the resulting modified oligonucleotides of the present invention comprise a sequence where at least 80% of all adenosine nucleotides are 2′-deoxy adenosine, at least 80% of all guanosine nucleotides are 2′-deoxy guanosine, at least 80% of all cytidine nucleotides are 2′-O-methyl cytidine, and at least 80% of all uridine nucleotides are 2′-O-methyl uridine. In a more preferred embodiment, the resulting modified oligonucleotides of the present invention comprise a sequence where at least 90% of all adenosine nucleotides are 2′-deoxy adenosine, at least 90% of all guanosine nucleotides are 2′-deoxy guanosine, at least 90% of all cytidine nucleotides are 2′-O-methyl cytidine, and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all guanosine nucleotides are 2′-deoxy guanosine, 100% of all cytidine nucleotides are 2′-O-methyl cytidine, and 100% of all uridine nucleotides are 2′-O-methyl uridine.

Under rGmH transcription conditions of the present invention, the transcription reaction mixture comprises 2′-OH guanosine triphosphates, 2′-O-methyl cytidine triphosphates, 2′-O-methyl uridine triphosphates, and 2′-O-methyl adenosine triphosphates. The modified oligonucleotides produced using the rGmH transcription mixtures of the present invention comprise substantially all 2′-OH guanosine, 2′-O-methyl cytidine, 2′-O-methyl uridine, and 2′-O-methyl adenosine. In a preferred embodiment, the resulting modified oligonucleotides comprise a sequence where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine. In a more preferred embodiment, the resulting modified oligonucleotides comprise a sequence where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine. In a most preferred embodiment, the resulting modified oligonucleotides comprise a sequence where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine. Under r/mGmH transcription conditions of the present invention, the transcription reaction mixture comprises 2′-O-methyl adenosine triphosphate, 2′-O-methyl cytidine triphosphate, 2′-O-methyl guanosine triphosphate, 2′-O-methyl uridine triphosphate and 2′-OH guanosine triphosphate. The resulting modified oligonucleotides produced using the r/mGmH transcription mixtures of the present invention comprise substantially all 2′-O-methyl adenosine, 2′-O-methyl cytidine, 2′-O-methyl guanosine, and 2′-O-methyl uridine, wherein the population of guanosine nucleotides has a maximum of about 10% 2′-OH guanosine. In a preferred embodiment, the resulting r/mGmH modified oligonucleotides of the present invention comprise a sequence where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are 2′-OH guanosine. In a more preferred embodiment, the resulting modified oligonucleotides comprise a sequence where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are 2′-OH guanosine. In a most preferred embodiment, the resulting modified oligonucleotides comprise a sequence where 100% of all adenosine nucleotides are 2′-O-methyl adenosine, 100% of all cytidine nucleotides are 2′-O-methyl cytidine, 90% of all guanosine nucleotides are 2′-O-methyl guanosine, and 100% of all uridine nucleotides are 2′-O-methyl uridine, and no more than about 10% of all guanosine nucleotides are 2′-OH guanosine.

Under fGmH transcription conditions of the present invention, the transcription reaction mixture comprises 2′-O-methyl adenosine triphosphates, 2′-O-methyl uridine triphosphates, 2′-O-methyl cytidine triphosphates, and 2′-F guanosine triphosphates. The modified oligonucleotides produced using the fGmH transcription conditions of the present invention comprise substantially all 2′-O-methyl adenosine, 2′-O-methyl uridine, 2′-O-methyl cytidine, and 2′-F guanosine. In a preferred embodiment, the resulting modified oligonucleotides comprise a sequence where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine. In a more preferred embodiment, the resulting modified oligonucleotides comprise a sequence where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine. In a most preferred embodiment, the resulting modified oligonucleotides comprise a sequence where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

Under dAmB transcription conditions of the present invention, the transcription reaction mixture comprises 2′-deoxy adenosine triphosphates, 2′-O-methyl cytidine triphosphates, 2′-O-methyl guanosine triphosphates, and 2′-O-methyl uridine triphosphates. The modified oligonucleotides produced using the dAmB transcription mixtures of the present invention comprise substantially all 2′-deoxy adenosine, 2′-O-methyl cytidine, 2′-O-methyl guanosine, and 2′-O-methyl uridine. In a preferred embodiment, the resulting modified oligonucleotides comprise a sequence where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine. In a more preferred embodiment, the resulting modified oligonucleotides comprise a sequence where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.

In each case, the transcription products can then be used as the library in the SELEX™ process to identify aptamers and/or to determine a conserved motif of sequences that have binding specificity to a given target. The resulting sequences are already partially stabilized, eliminating this step from the process to arrive at an optimized aptamer sequence and giving a more highly stabilized aptamer as a result. Another advantage of the 2′-OMe SELEX™ process is that the resulting sequences are likely to have fewer 2′-OH nucleotides required in the sequence, possibly none. To the extent 2′OH nucleotides remain they can be removed by performing post-SELEX™ modifications.

As described below, lower but still useful yields of transcripts fully incorporating 2′ substituted nucleotides can be obtained under conditions other than the optimized conditions described above. For example, variations to the above transcription conditions include:

The HEPES buffer concentration can range from 0 to 1 M. The present invention also contemplates the use of other buffering agents having a pKa between 5 and 10 including, for example, Tris-hydroxymethyl-aminomethane.

The DTT concentration can range from 0 to 400 mM. The methods of the present invention also provide for the use of other reducing agents including, for example, mercaptoethanol.

The spermidine and/or spermine concentration can range from 0 to 20 mM.

The PEG-8000 concentration can range from 0 to 50 % (w/v). The methods of the present invention also provide for the use of other hydrophilic polymer including, for example, other molecular weight PEG or other polyalkylene glycols.

The Triton X-100 concentration can range from 0 to 0.1% (w/v). The methods of the present invention also provide for the use of other non-ionic detergents including, for example, other detergents, including other Triton-X detergents.

The MgCl₂ concentration can range from 0.5 mM to 50 mM. The MnCl₂ concentration can range from 0.15 mM to 15 mM. Both MgCl₂ and MnCl₂ must be present within the ranges described and in a preferred embodiment are present in about a 10 to about 3 ratio of MgCl₂:MnCl₂, preferably, the ratio is about 3-5:1, more preferably, the ratio is about 3-4:1.

The 2′-OMe NTP concentration (each NTP) can range from 5 μM to 5 mM.

The 2′-OH GTP concentration can range from 0 μM to 300 μM.

The 2′-OH GMP concentration can range from 0 to 5 mM.

The pH can range from pH 6 to pH 9. The methods of the present invention can be practiced within the pH range of activity of most polymerases that incorporate modified nucleotides. In addition, the methods of the present invention provide for the optional use of chelating agents in the transcription reaction condition including, for example, EDTA, EGTA, and DTT.

In some embodiments aptamer therapeutics of the present invention have great affinity and specificity to their targets while reducing the deleterious side effects from non-naturally occurring nucleotide substitutions if the aptamer therapeutics break down in the body of patients or subjects. In some embodiments, the therapeutic compositions containing the aptamer therapeutics of the present invention are free of or have a reduced amount of fluorinated nucleotides.

The aptamers of the present invention can be synthesized using any oligonucleotide synthesis techniques known in the art including solid phase oligonucleotide synthesis techniques well known in the art (see, e.g., Froehler et al., Nucl. Acid Res. 14:5399-5467 (1986) and Froehler et al., Tet. Lett. 27:5575-5578 (1986)) and solution phase methods such as triester synthesis methods (see, e.g., Sood et al., Nucl. Acid Res. 4:2557 (1977) and Hirose et al., Tet. Lett., 28:2449 (1978)).

Aptamers Having Immunostimulatory Motifs

Recognition of bacterial DNA by the vertebrate immune system is based on the recognition of unmethylated CG dinucleotides in particular sequence contexts (“CpG motifs”). One receptor that recognizes such a motif is Toll-like receptor 9 (“TLR 9”), a member of a family of Toll-like receptors (˜10 members) that participate in the innate immune response by recognizing distinct microbial components. TLR 9 binds unmethylated oligodeoxynucleotide (“ODN”) CpG sequences in a sequence-specific manner. The recognition of CpG motifs triggers defense mechanisms leading to innate and ultimately acquired immune responses. For example, activation of TLR 9 in mice induces activation of antigen presenting cells, up regulation of MHC class I and II molecules and expression of important co-stimulatory molecules and cytokines including IL-12 and IL-23. This activation both directly and indirectly enhances B and T cell responses, including robust up regulation of the TH1 cytokine IFN-gamma. Collectively, the response to CpG sequences leads to: protection against infectious diseases, improved immune response to vaccines, an effective response against asthma, and improved antibody-dependent cell-mediated cytotoxicity. Thus, CpG ODNs can provide protection against infectious diseases, function as immuno-adjuvants or cancer therapeutics (monotherapy or in combination with a mAb or other therapies), and can decrease asthma and allergic response.

In some embodiments, aptamers of the present invention comprise one or more immunostimulatory sequence such as a CpG motif. In particular embodiments, aptamers comprising immunostimulatory motifs are provided for treating, preventing or ameliorating cancer, infectious disease, asthma or allergy. Aptamers of the present invention comprising one or more CpG or other immunostimulatory sequences can be identified or generated by a variety of strategies using, e.g., the SELEX™ process described herein. In general the strategies can be divided into two groups. In group one, the strategies are directed to identifying or generating aptamers comprising both a CpG motif or other immunostimulatory sequence as well as a binding site for a target, where the target (hereinafter “non-CpG target”) is a target other than one known to recognize CpG motifs or other immunostimulatory sequences and known to stimulates an immune response upon binding to a CpG motif. In some embodiments of the invention the non-CpG target is a target that relates to the growth of cancers tissue and/or tumors. The first strategy of this group comprises performing SELEX™ to obtain an aptamer to a specific non-CpG target, preferably a target where a repressed immune response is relevant to disease development, using an oligonucleotide pool wherein a CpG motif has been incorporated into each member of the pool as, or as part of, a fixed region, e.g., in some embodiments the randomized region of the pool members comprises a fixed region having a CpG motif incorporated therein, and identifying an aptamer comprising a CpG motif. The second strategy of this group comprises performing SELEX™ to obtain an aptamer to a specific non-CpG target preferably a target, where a repressed immune response is relevant to disease development, and following selection appending a CpG motif to the 5′ and/or 3′ end or engineering a CpG motif into a region, preferably a non-essential region, of the aptamer. The third strategy of this group comprises performing SELEX™ to obtain an aptamer to a specific non-CpG target, preferably a target, e.g. where a repressed immune response is relevant to disease development, wherein during synthesis of the pool the molar ratio of the various nucleotides is biased in one or more nucleotide addition steps so that the randomized region of each member of the pool is enriched in CpG motifs, and identifying an aptamer comprising a CpG motif. The fourth strategy of this group comprises performing SELEX™ to obtain an aptamer to a specific non-CpG target, preferably a target, where a repressed immune response is relevant to disease development, and identifying an aptamer comprising a CpG motif. The fifth strategy of this group comprises performing SELEX™ to obtain an aptamer to a specific non-CpG target, preferably a target, where a repressed immune response is relevant to disease development, and identifying an aptamer which, upon binding, stimulates an immune response but which does not comprise a Cpq motif.

In group two, the strategies are directed to identifying or generating aptamers comprising a CpG motif and/or other sequences that are bound by the receptors for the CpG motifs (e.g., TLR9 or the other toll-like receptors) and upon binding stimulate an immune response. The first strategy of this group comprises performing SELEX™ to obtain an aptamer to a target known to bind to CpG motifs or other immunostimulatory sequences and upon binding stimulate an immune response using an oligonucleotide pool wherein a CpG motif has been incorporated into each member of the pool as, or as part of, a fixed region, e.g., in some embodiments the randomized region of the pool members comprise a fixed region having a CpG motif incorporated therein, and identifying an aptamer comprising a CpG motif. The second strategy of this group comprises performing SELEX™ to obtain an aptamer to a target known to bind to CpG motifs or other immunostimulatory sequences and upon binding stimulate an immune response and then appending a CpG motif to the 5′ and/or 3′ end or engineering a CpG motif into a region, preferably a non-essential region, of the aptamer. The third strategy of this group comprises performing SELEX™ to obtain an aptamer to a target known to bind to CpG motifs or other immunostimulatory sequences and upon binding stimulate an immune response wherein during synthesis of the pool, the molar ratio of the various nucleotides-is biased in one or more nucleotide addition steps so that the randomized region of each member of the pool is enriched in CpG motifs, and identifying an aptamer comprising a CpG motif. The fourth strategy of this group comprises performing SELEX™ to obtain an aptamer to a target known to bind to CpG motifs or other immunostimulaory sequences and upon binding stimulate an immune response and identifying an aptamer comprising a CpG motif. The fifth strategy of this group comprises performing SELEX™ to obtain an aptamer to a target known to bind to CpG motifs or other immunostimulatory sequences, and identifying an aptamer which upon binding, stimulate an immune response but which does not comprise a CpG motif.

A variety of different classes of CpG motifs have been identified, each resulting upon recognition in a different cascade of events, release of cytokines and other molecules, and activation of certain cell types. See, e.g., CpG Motifs in Bacterial DNA and Their Immune Effects, Annu. Rev. Immunol. 2002, 20:709-760, incorporated herein by reference. Additional immunostimulatory motifs are disclosed in the following U.S. Patents, each of which is incorporated herein by reference: U.S. Pat. No. 6,207,646; U.S. Pat. No. 6,239,116; U.S. Pat. No. 6,429,199; U.S. Pat. No. 6,214,806; U.S. Pat. No. 6,653,292; U.S. Pat. No. 6,426,434; U.S. Pat. No. 6,514,948 and U.S. Pat. No. 6,498,148. Any of these CpG or other immunostimulatory motifs can be incorporated into an aptamer of the invention. The choice of aptamers is dependent on the disease or disorder to be treated. Preferred immunostimulatory motifs are as follows (shown 5′ to 3′ left to right) wherein “r” designates a purine, “y” designates a pyrimidine, and “X” designates any nucleotide: AACGTTCGAG (SEQ ID NO: 4); AACGTT; ACGT, rCGy; rrCGyy, XCGX, XXCGXX, and X₁X₂CGY₁Y₂ wherein X₁ is G or A, X₂ is not C, Y₁ is not G and Y₂ is preferably T.

In those instances where a CpG motif is incorporated into an aptamer that binds to a specific target other than a target known to bind to CpG motifs and upon binding stimulate an immune response (a “non-CpG target”), the CpG is preferably located in a non-essential region of the aptamer. Non-essential regions of aptamers can be identified by site-directed mutagenesis, deletion analyses and/or substitution analyses. However, any location that does not significantly interfere with the ability of the aptamer to bind to the non-CpG target may be used. In addition to being embedded within the aptamer sequence, the CpG motif may be appended to either or both of the 5′ and 3′ ends or otherwise attached to the aptamer. Any location or means of attachment may be used so long as the ability of the aptamer to bind to the non-CpG target is not significantly interfered with.

As used herein, “stimulation of an immune response” can mean either (1) the induction of a specific response (e.g., induction of a Th1 response) or of the production of certain molecules or (2) the inhibition or suppression of a specific response (e.g., inhibition or suppression of the Th2 response) or of certain molecules.

Aptamer Therapeutics

It is important that the pharmacokinetic properties for all oligonucleotide-based therapeutics, including aptamers, be tailored to match the desired pharmaceutical application. While aptamers directed against extracellular targets do not suffer from difficulties associated with intracellular delivery (as is the case with antisense and RNAi-based therapeutics), such aptamers must be able to be distributed to target organs and tissues, and remain in the body (unmodified) for a period of time consistent with the desired dosing regimen. Early work on nucleic acid-based therapeutics has shown that, while unmodified oligonucleotides are degraded rapidly by nuclease digestion, protective modifications at the 2′-position of the sugar, and use of inverted terminal cap structures, e.g., [3′-3′dT], dramatically improve nucleic acid stability in vitro and in vivo (Green, et al. (1995), Chem. Biol. 2(10): 683-95; Jellinek, et al. (1995), Biochemistry 34(36): 11363-72; Ruckman, et al. (1998), J. Biol. Chem. 273(32): 20556-67; Uhlmann, et al. (2000), Methods Enzymol. 313: 268-84). In some SELEX™ selections (i.e., SELEX™ experiments or SELEXions), starting pools of nucleic acids from which aptamers are selected are typically pre-stabilized by chemical modification, for example by incorporation of 2′-fluoropyrimidine (2′-F) substituted nucleotides, to enhance resistance of aptamers against nuclease attack. Aptamers incorporating 2′-O-methylpurine (2′-O-Me purine) substituted nucleotides have also been developed through post-SELEX™ modification steps or, more recently, by enabling synthesis of 2′-O-Me-containing random sequence libraries as an integral component of the SELEX™ process itself, as described above.

In addition to clearance by nucleases, oligonucleotide therapeutics are subject to elimination via renal filtration. As such, a nuclease-resistant oligonucleotide administered intravenously exhibits an in vivo half-life of <10 min, unless filtration can be blocked. This can be accomplished by either facilitating rapid distribution out of the blood stream into tissues or by increasing the apparent molecular weight of the oligonucleotide above the effective size cut-off for glomerular filtration. Conjugation of aptamer therapeutics to a PEG polymer (PEGylation) can dramatically lengthen residence times of aptamers in circulation, thereby decreasing dosing frequency and enhancing effectiveness against vascular targets. Previous work in animals has examined the plasma pharmacokinetic properties of PEG-conjugated aptamers (Reyderman and Stavchansky (1998), Pharmaceutical Research 15(6): 904-10; Watson, et al. (2000), Antisense Nucleic Acid Drug Dev. 10(2): 63-75). However, relatively little is known about the capacity of either unconjugated or PEGylated aptamers to escape the vasculature and distribute to organs and tissues in vivo.

Modulation of Pharmacokinetics and Biodistribution of Aptamer Therapeutics

The present invention provides materials and methods to effect the pharmacokinetics of aptamer compositions, and, in particular, the ability to tune (i.e., the “tunability”) aptamer pharmacokinetics. The tunability of aptamer pharmacokinetics is achieved through conjugation of modifying moieties to the aptamer and/or incorporation of modified nucleotides to alter the chemical composition of the nucleic acid. The ability to tune aptamer pharmacokinetics is used in the improvement of existing therapeutic applications, or alternatively, in the development of new therapeutic applications. For example, in some embodiments involving anti-neoplastic or acute care settings where rapid drug clearance or turn-off may be desired, it is desirable to decrease the residence times of aptamers in the circulation. In other embodiments, e.g., maintenance therapies where systemic circulation of a therapeutic is desired, it may be desirable to increase the residence times of aptamers in circulation.

In addition, the tunability of aptamer pharmacokinetics is used to modify the biodistribution of an aptamer therapeutic in a subject. For example, in some embodiments it is desirable to alter the biodistribution of an aptamer therapeutic to target a particular type of tissue or a specific organ (or set of organs). In these embodiments, the aptamer therapeutic is formulated to preferentially accumulate in a specific tissue (s) or organ(s). In other embodiments, it is desirable to target tissues displaying a cellular marker or a symptom associated with a given disease, cellular injury or other abnormal pathology, such that the aptamer therapeutic preferentially accumulates in the affected tissue. For example, as described herein, PEGylation of an aptamer therapeutic (e.g. PEGylation with a 20 kDa PEG polymer as shown in Example 10) is used to target inflamed tissues, such that the PEGylated aptamer therapeutic preferentially accumulates in inflamed tissue.

The pharmacokinetic and biodistribution of aptamer therapeutics (e.g., aptamer conjugates or aptamers having altered chemistries, such as modified nucleotides) are determined by monitoring a variety of parameters. Such parameters include, for example, the half-life (t1/2), the plasma clearance (C1), the volume of distribution (Vss), the area under the concentration-time curve (AUC), maximum observed serum or plasma concentration (Cmax), and the mean residence time (MRT) of an aptamer composition. As used herein, the term “AUC” refers to the area under the plot of the plasma concentration of an aptamer therapeutic versus the time after aptamer administration. The AUC value is used to estimate the bioavailability (i.e., the percentage of administered aptamer therapeutic in the circulation after aptamer administration; e.g., to determine the bioavailability of a given route of administration, i.e. subcutaneous, the ratio Fsubcut=(AUCsubcut/AUCiv) is the ratio of the AUC for the subcutaneous route to the AUC for the intravenous route) and/or total clearance (Cl) (i.e., the rate at which the aptamer therapeutic is removed from circulation) of a given aptamer therapeutic. The volume of distribution (VSS) relates the plasma concentration of an aptamer therapeutic to the amount of aptamer present in the body. The larger the Vss, the greater the proportion of an administered dose of aptamer that is found outside of the plasma (i.e., the more extravasation).

The pharmacokinetic and biodistribution properties of phosphorothioate-containing antisense oligonucleotides, which clear rapidly from circulation, and distribute into tissues (where elimination occurs slowly, as a result of metabolic degradation) are described in the art. (See e.g., Srinivasan and Iversen (1995), J. Clin. Lab. Anal. 9(2): 129-37; Agrawal and Zhang (1997), Ciba Found. Symp. 209: 60-75, discussion 75-8; Akhtar and Agrawal (1997), Trends Pharmacol. Sci. 18(1): 12-8; Crooke (1997), Adv. Pharmacol. 40: 1-49; Grindel, et al. (1998), Antisense Nucleic Acid Drug Dev. 8(1): 43-52; Monteith and Levin (1999), Toxicol. Pathol. 27(1): 8-13; Peng, et al. (2001), Antisense Nucleic Acid Drug Dev. 11(1): 15-27). Early studies involving antisense oligonucleotides have explored the effects of various conjugation chemistries on pharmacokinetics and biodistribution, with the ultimate goal of increasing delivery of antisense molecules to their sites of action inside cells or within certain tissue types in vivo (Antopolsky, et al. (1999), Bioconjug. Chem. 10(4): 598-606; Zubin, et al. (1999), FEBS Lett. 456(1): 59-62; Astriab-Fisher, et al. (2000),. Biochem. Pharmacol. 60(1): 83-90; Lebedeva, et al. (2000), Eur. J. Pharm. Biopharm. 50(1): 101-19; Manoharan (2002), Antisense Nucleic Acid Drug Dev. 12(2): 103-28). For example, conjugation with cholesterol has been reported to increase the circulation half-life of antisense oligonucleotides, most likely through association with plasma lipoproteins, and promoting hepatic uptake (de Smidt, et al. (1991), Nucleic Acids Res. 19(17): 4695-4700). Early work involving antisense oligonucleotides has indicated that nonspecific protein-binding interactions play an important role in the rapid loss of phosphorothioate-containing antisense oligonucleotide from circulation and distribution to tissues (See e.g., Srinivasan and Iversen (1995), J. Clin. Lab. Anal. 9(2): 129-37; Agrawal and Zhang (1997), Ciba Found. Symp. 209: 60-75, discussion 75-8; Akhtar and Agrawal (1997), Trends Pharmacol. Sci 18(1): 12-8; Crooke (1997), Adv. Pharmacol. 40: 1-49; Grindel, et al. (1998), Antisense Nucleic Acid Drug Dev. 8(1): 43-52; Monteith and Levin (1999), Toxicol. Pathol. 27(1): 8-13; Peng, et al. (2001), Antisense Nucleic Acid Drug Dev. 11(1): 15-27).

In contrast to antisense oligonucleotides, aptamers are generally longer (30-40 vs. 10-20 nucleotides), possess different types of chemical modifications (sugar modifications, e.g., 2′-F, 2′-O-Me, 2′-NH2, vs. backbone modifications), and assume complex tertiary structures that are more resistant to degradation. Aptamers are, in many respects, more structurally similar to the three dimensional forms of globular proteins than to nucleic acids. Given these considerable differences, the in vivo disposition of aptamers is not readily predictable from antisense results.

More recently, delivery peptides for carrying large polar macromolecules, including oligonucleotides, across cellular membranes have also been explored as a means to augment in vivo the range for application of antisense and other therapeutics. Examples of these conjugates include a 13-amino acid fragment (Tat) of the HIV Tat protein (Vives, et al. (1997), J. Biol. Chem. 272(25): 16010-7), a 16-amino acid sequence derived from the third helix of the Drosophila antennapedia (Ant) homeotic protein (Pietersz, et al. (2001), Vaccine 19(11-12): 1397-405), and short, positively charged cell-permeating peptides composed of polyarginine (Arg7) (Rothbard, et al. (2000), Nat. Med. 6(11): 1253-7; Rothbard, J et al. (2002), J. Med. Chem. 45(17): 3612-8). For example, the TAT peptide is described in U.S. Pat. Nos. 5,804,604 and 5,674,980.

The present invention provides materials and methods to modulate, in a controlled manner, the pharmacokinetics and biodistribution of stabilized aptamer compositions in vivo by conjugating an aptamer to a modulating moiety such as a small molecule, peptide, or polymer terminal group, and/or by incorporating modified nucleotides into an aptamer. Pharmacokinetics and biodistribution of aptamer conjugates in biological samples are quantified radiometrically and by a hybridization-based dual probe capture assay with enzyme-linked fluorescent readout. As described in the Examples section, conjugation of a modifying moiety and/or altering nucleotide(s) chemical composition alter fundamental aspects of aptamer residence time in circulation and distribution to tissues.

Aptamers are conjugated to a variety of modifying moieties, such as, for example, high molecular weight polymers, e.g., PEG, peptides, e.g., Tat, Ant and Arg7, and small molecules, e.g., lipophilic compounds such as cholesterol. As shown in the Examples provided herein, a mixed composition aptamer containing both 2′F and 2′-O-Me stabilizing modifications persisted significantly longer in the blood stream than did a fully 2′-O-methylated composition. Among the conjugates prepared according to the materials and methods of the present invention, in vivo properties of aptamers were altered most profoundly by complexation with PEG groups. Furthermore, complexation of this chemically modified oligonucleotide with a 20 kDa PEG polymer proved nearly as effective as a 40 kDa PEG polymer in preventing renal filtration of aptamers. Though the primary effect of PEGylation was on aptamer clearance, the prolonged systemic exposure afforded by presence of the 20 kDa moiety facilitated distribution of aptamer to tissues, particularly those of highly perfused organs.

Overall, effects on aptamer pharmacokinetics and tissue distribution produced by low molecular weight modifying moieties, including cholesterol and cell-permeating peptides were less pronounced than those produced as a result of PEGylation or modification of nucleotides (e.g., an altered chemical composition). As shown in the Examples provided herein, an aptamer conjugated to cholesterol, referred to herein as, ARC155, showed more rapid plasma clearance relative to unconjugated aptamer, and a large volume of distribution, which suggests some degree of aptamer extravasation. This result appears to contrast published data demonstrating the capacity of a cholesterol tag to significantly prolong the plasma half-life of an antisense oligonucleotide (de Smidt, et al. (1991), Nucleic Acids Res. 19(17): 4695-4700). While not intending to be bound by theory, the results provided in the Examples herein, may suggest that cholesterol-mediated associations with plasma lipoproteins, postulated to occur in the case of the antisense conjugate, are precluded in the particular context of the ARC155 folded structure, and/or relate to aspect of the lipophilic nature of the cholesterol group. Like cholesterol, the presence of a Tat peptide tag promoted clearance of aptamer from the blood stream, with comparatively high levels of conjugate appearing in the kidneys at 48 hrs. Other peptides (e.g., Ant, Arg7) that have been reported in the art to mediate passage of macromolecules across cellular membranes in vitro, did not appear to promote aptamer clearance from circulation. However, like Tat, the Ant conjugate significantly accumulated in the kidneys relative to other aptamers. While not intending to be bound by theory, it is possible that unfavorable presentation of the Ant and Arg7 peptide modifying moieties in the context of three dimensionally folded aptamers in vivo impaired the ability of these peptides to influence aptamer transport properties.

Prior to the invention described herein, little was known concerning the pharmacokinetics and biodistribution of oligonucleotides with a 2′-O-Me chemical composition (Tavitian, et al. (1998), Nat. Med. 4(4): 467-71). For several reasons, incorporation of 2′-O-Me substitutions is a particularly attractive means to stabilize aptamers against nuclease attack. One attribute is safety: 2′-O-methylation is known as a naturally occurring and abundant chemical modification in eukaryotic ribosomal and cellular RNAs. Human rRNAs are estimated to contain roughly one hundred 2′-O-methylated sugars per ribosome (Smith and Steitz (1997), Cell 89(5): 669-72). Thus, aptamer compositions incorporating 2′-O-Me substitutions are expected to be non-toxic. In support of this view, in vitro and in vivo studies indicate that 2′-O-Me nucleotides are not readily polymerized by human DNA polymerases (a or y), or by human DNA primase, and thus, pose a low risk for incorporation into genomic DNA (Richardson, et al. (2000), Biochem. Pharmacol. 59(9): 1045-52; Richardson, et al. (2002), Chem. Res. Toxicol. 15(7): 922-6). Additionally, from a cost of goods perspective, pricing per gram for synthesis of 2′-O-Me containing oligonucleotides is less than the pricing per gram for both 2′-F and 2′-OH containing RNAs.

The Examples provided herein used a mixed 2′F/2′-O-Me composition aptamer, referred to herein as ARC83, as the basis for preparing conjugated aptamers and aptamers having altered chemistries (e.g., containing modified nucleotides). The unconjugated test aptamer, ARC83, which incorporates both 2′-F and 2′-O-Me stabilizing chemistries, is typical of current generation aptamers as it exhibits a high degree of nuclease stability in vitro and in vivo. Compared to the mixed 2′F/2′-O-Me composition aptamer (ARC83), a fully 2′-O-Me modified aptamer, referred to herein as ARC159, displayed rapid loss from plasma (i.e., rapid plasma clearance) and a rapid distribution into tissues, primarily into the kidney, and rapid urinary elimination.

While not intending to be bound by a particular theory, levels of full-length ARC 159 above background were detected in several tissues, kidney, liver, and spleen, even at 48 hrs after dosing, possibly due to the extreme robustness of the fully 2′-O-Me aptamer towards nuclease digestion. Consistent with its plasma clearance profile and distribution to the kidney, the fully 2′-O-Me aptamer, ARC159, was eliminated rapidly via the urine.

When expressed as percent of administered dose, all aptamers or conjugates examined herein showed significant levels of distribution to kidney, liver, and gastrointestinal tract. When corrected for organ/tissue weight, highest mass-normalized concentrations of aptamers were seen in highly perfused organs (kidneys, liver, spleen, heart, lungs) and unexpectedly, mediastinal lymph nodes. Since aptamers are bioavailable (up to 80 %) following subcutaneous injection (Tucker et al., (1999), J. Chromatography B. 732: 203-212), they are expected to have access to targets in the lymphatic system through this route of administration. Ready access to the lymphatics via intravenous dosing is of interest from the standpoint of developing aptamer therapeutics for infectious disease indications such as HIV/AIDS. Thus, aptamer therapeutics conjugated to modifying moieties and aptamers having altered chemistries (e.g., including modified nucleotides) will be useful in the treatment of infectious diseases such as HIV/AIDS.

Consistent with its enhanced plasma pharmacokinetics, the concentration of 20 kDa PEGylated aptamer (ARC120) detected in highly perfused organs was higher than for the other aptamers that were assayed. As a general trend, aptamer concentrations measured in the kidneys decreased with time, with exception of ARC120, where concentrations remained roughly constant over time. Conversely, in liver concentrations of all aptamers remained roughly constant, except for ARC120, whose levels decreased with time. These differences may be understood in terms of the extended plasma half-life of the 20 kDa PEG conjugate and its increased uptake in highly perfused organs. Though the main effect of complexation with a 20 kDa PEG modifying moiety was to retard renal filtration of the aptamer conjugate, the comparatively high concentrations of the 20 kDa PEG conjugate measured in well-perfused organs, relative to other aptamers or conjugates, suggests that PEGylation can modulate aptamer distribution to tissues, as well as promote extended plasma half-life (t1/2).

While not intending to be bound by theory, it is believed that prolonged residence in the blood stream increases exposure of conjugated aptamer to tissues, leading to enhanced uptake. This effect is most pronounced in the case of highly perfused organs. The presence of aptamer in residual blood may contribute to, but is unlikely to account entirely for, the increased levels of the 20 kDa aptamer conjugate in perfused organs shown herein. The enhanced distribution of PEGylated aptamer to perfused organs represents extravasation, as suggested by experiments in mice dosed with tritiated ARC120 (20 kDa PEG conjugate) where [³H] signal was seen in cells of both the liver and kidney (See Examples provided below). Early work on aptamer therapeutics focused primarily on development of aptamers complexed with higher molecular weight (40 kDa) PEG species in an effort to avoid renal filtration (Reyderman and Stavchansky (1998), Pharmaceutical Research 15(6): 904-10; Tucker et al., (1999), J. Chromatography B. 732: 203-212; Watson, et al. (2000), Antisense Nucleic Acid Drug Dev. 10(2): 63-75; Carrasquillo, et al. (2003), Invest. Ophthalmology Vis. Sci. 44(1): 290-9). The present invention indicates that complexation with a smaller, 20 kDa, PEG polymer sufficiently protects aptamer-based drugs from renal filtration for many therapeutic indications. Smaller PEGs (e.g., 10 kDa to 20 kDa PEG moieties) also provide the collateral benefits of ease of synthesis and reduced cost of goods, as compared to larger PEGs.

PEG-Derivatized Nucleic Acids

Derivatization of nucleic acids with high molecular weight non-immunogenic polymers has the potential to alter the pharmacokinetic and pharmacodynamic properties of nucleic acids making them more effective therapeutic agents. Favorable changes in activity can include increased resistance to degradation by nucleases, decreased filtration through the kidneys, decreased exposure to the immune system, and altered distribution of the therapeutic through the body.

The aptamer compositions of the invention may be derivatized with polyalkylene glycol (PAG) moieties. Typical polymers used in the invention include poly(ethylene glycol) (PEG), also known as or poly(ethylene oxide) (PEO) and polypropylene glycol (including poly isopropylene glycol). Additionally, random or block copolymers of different alkylene oxides (e.g., ethylene oxide and propylene oxide) can be used in many applications. In its most common form, a polyalkylene glycol, such as PEG, is a linear polymer terminated at each end with hydroxyl groups: HO—CH₂CH₂O—(CH₂CH₂O)ₙ—CH₂CH₂—OH. This polymer, alpha-, omega-dihydroxylpoly(ethylene glycol), can also be represented as HO-PEG-OH, where it is understood that the—PEG—symbol represents the following structural unit: —CH₂CH₂— (CH₂CH₂O)ₙ—CH₂CH₂— where n typically ranges from about 4 to about 10,000.

As shown, the PEG molecule is di-functional and is sometimes referred to as “PEG diol.” The terminal portions of the PEG molecule are relatively non-reactive hydroxyl moieties, the —OH groups, that can be activated, or converted to functional moieties, for attachment of the PEG to other compounds at reactive sites on the compound. Such activated PEG diols are referred to herein as bi-activated PEGs. For example, the terminal moieties of PEG diol have been functionalized as active carbonate ester for selective reaction with amino moieties by substitution of the relatively nonreactive hydroxyl moieties, —OH, with succinimidyl active ester moieties from N-hydroxy succinimide.

In many applications, it is desirable to cap the PEG molecule on one end with an essentially non-reactive moiety so that the PEG molecule is mono-functional (or mono-activated). In the case of protein therapeutics which generally display multiple reaction sites for activated PEGs, bi-functional activated PEGs lead to extensive cross-linking, yielding poorly functional aggregates. To generate mono-activated PEGs, one hydroxyl moiety on the terminus of the PEG diol molecule typically is substituted with non-reactive methoxy end moiety, —OCH3. The other, un-capped terminus of the PEG molecule typically is converted to a reactive end moiety that can be activated for attachment at a reactive site on a surface or a molecule such as a protein.

PAGs are polymers which typically have the properties of solubility in water and in many organic solvents, lack of toxicity, and lack of immunogenicity. One use of PAGs is to covalently attach the polymer to insoluble molecules to make the resulting PAG-molecule “conjugate” soluble. For example, it has been shown that the water-insoluble drug paclitaxel, when coupled to PEG, becomes water-soluble. Greenwald, et al., J. Org. Chem., 60:331-336 (1995). PAG conjugates are often used not only to enhance solubility and stability but also to prolong the blood circulation half-life of molecules.

In some embodiments, polyalkylated compounds of the invention are typically between 5 and 80 kD in size. Other PAG compounds of the invention are between 10 and 80 kD in size. Still other PAG compounds of the invention are between 10 and 60 kD in size. For example, a PAG polymer may be at least 5, 10, 20, 30, 40, 50, 60, or 80 kD in size. In some embodiments, a PAG polymer of the invention is no more than 30, 20, 10 or 5 kD in size. Such polymers can be linear or branched.

In contrast to biologically-expressed protein therapeutics, nucleic acid therapeutics are typically chemically synthesized from activated monomer nucleotides. PEG-nucleic acid conjugates may be prepared by incorporating the PEG using the same iterative monomer synthesis. For example, PEGs activated by conversion to a phosphoramidite form can be incorporated into solid-phase oligonucleotide synthesis. Alternatively, oligonucleotide synthesis can be completed with site-specific incorporation of a reactive PEG attachment site. Most commonly this has been accomplished by addition of a free primary amine at the 5′-terminus (incorporated using a modifier phosphoramidite in the last coupling step of solid phase synthesis). Using this approach, a reactive PEG (e.g., one which is activated so that it will react and form a bond with an amine) is combined with the purified oligonucleotide and the coupling reaction is carried out in solution.

The ability of PEG conjugation to alter the biodistribution of a therapeutic is related to a number of factors including the apparent size (e.g., as measured in terms of hydrodynamic radius) of the conjugate. Larger conjugates (>10 kDa) are known to more effectively block filtration via the kidney and to consequently increase the serum half-life of small macromolecules (e.g., peptides, antisense oligonucleotides). The ability of PEG conjugates to block filtration has been shown to increase with PEG size up to approximately 50 kDa (further increases have minimal beneficial effect as half life becomes defined by macrophage-mediated metabolism rather than elimination via the kidneys).

Production of high molecular weight PEGs (>10 kDa) can be difficult, inefficient, and expensive. As a route towards the synthesis of high molecular weight PEG-nucleic acid conjugates, previous work has been focused towards the generation of higher molecular weight activated PEGs. One method for generating such molecules involves the formation of a branched activated PEG in which two or more PEGs are attached to a central core carrying the activated group. The terminal portions of these higher molecular weight PEG molecules, i.e., the relatively non-reactive hydroxyl (-OH) moieties, can be activated, or converted to functional moieties, for attachment of one or more of the PEGs to other compounds at reactive sites on the compound. Branched activated PEGs will have more than two termini, and in cases where two or more termini have been activated, such activated higher molecular weight PEG molecules are referred to herein as, multi-activated PEGs. In some cases, not all termini in a branch PEG molecule are activated. In cases where any two termini of a branch PEG molecule are activated, such PEG molecules are referred to as bi-activated PEGs. In some cases where only one terminus in a branch PEG molecule is activated, such PEG molecules are referred to as mono-activated. As an example of this approach, activated PEG prepared by the attachment of two monomethoxy PEGs to a lysine core which is subsequently activated for reaction has been described (Harris et al., Nature, vol.2: 214-221, 2003).

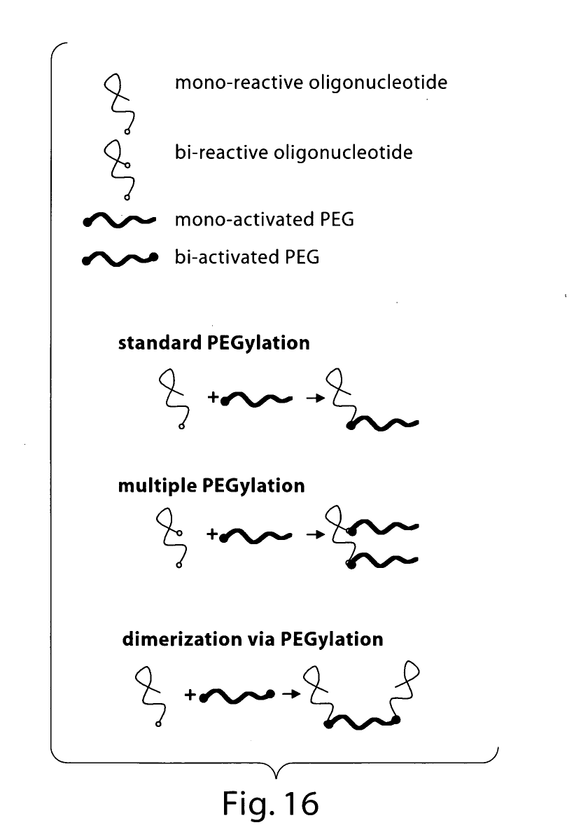

The present invention provides another cost effective route to the synthesis of high molecular weight PEG-nucleic acid (preferably, aptamer) conjugates including multiply PEGylated nucleic acids (as illustrated, e.g., in FIG. 16). The present invention also encompasses PEG-linked multimeric oligonucleotides, e.g., dimerized aptamers (as also illustrated, e.g., in FIG. 26). The present invention also relates to high molecular weight compositions where a PEG stabilizing moiety is a linker which separates different portions of an aptamer, e.g., the PEG is conjugated within a single aptamer sequence, such that the linear arrangement of the high molecular weight aptamer composition is, e.g., nucleic acid—PEG—nucleic acid—PEG—nucleic acid, or, e.g., PEG—nucleic acid—PEG—nucleic acid—PEG—nucleic acid.

High molecular weight compositions of the invention include those having a molecular weight of at least 10 kD. High molecular weight compositions of the invention typically have a molecular weight between 10 and 80 kD in size. High molecular weight compositions of the invention are at least 10, 20, 30, 40, 50, 60, or 80 kD in size.

A stabilizing moiety is a molecule, or portion of a molecule, which improves pharmacokinetic and pharmacodynamic properties of the high molecular weight aptamer compositions of the invention. In some cases, a stabilizing moiety is a molecule or portion of a molecule which brings two or more aptamers, or aptamer domains, into proximity, or provides decreased overall rotational freedom of the high molecular weight aptamer compositions of the invention. A stabilizing moiety can be a polyalkylene glycol, such a polyethylene glycol, which can be linear or branched, a homopolymer or a heteropolymer. Other stabilizing moieties include polymers such as peptide nucleic acids (PNA). A stabilizing moiety can be an integral part of an aptamer composition, i.e., it is covalently bonded to the aptamer. A stabilizing moiety can also be a modified nucleotide incorporated into an aptamer of the invention, e.g., a 2′ modification or a modified linkage (e.g., modified phosphate linkage).

Compositions of the invention include high molecular weight aptamer compositions in which two or more nucleic acid moieties are covalently conjugated to at least one polyalkylene glycol moiety. The polyalkylene glycol moieties serve as stabilizing moieties. In compositions where a polyalkylene glycol moiety is covalently bound at either end to an aptamer, such that the polyalkylene glycol joins the nucleic acid moieties together in one molecule, the polyalkylene glycol is said to be a linking moiety. In such compositions, the primary structure of the covalent molecule includes the linear arrangement nucleic acid-PAG-nucleic acid. One example is a composition having the primary structure nucleic acid-PEG-nucleic acid. Another example is a linear arrangement of: nucleic acid—PEG—nucleic acid—PEG—nucleic acid.

To produce the nucleic acid—PEG—nucleic acid conjugate, the nucleic acid is originally synthesized such that it bears a single reactive site (e.g., it is mono-activated). In a preferred embodiment, this reactive site is an amino group introduced at the 5′-terminus by addition of a modifier phosphoramidite as the last step in solid phase synthesis of the oligonucleotide. Following deprotection and purification of the modified oligonucleotide, it is reconstituted at high concentration in a solution that minimizes spontaneous hydrolysis of the activated PEG. In a preferred embodiment, the concentration of oligonucleotide is 1 mM and the reconstituted solution contains 200 mM NaHCO3-buffer, pH 8.3. Synthesis of the conjugate is initiated by slow, step-wise addition of highly purified bi-functional PEG. In a preferred embodiment, the PEG diol is activated at both ends (bi-activated) by derivatization with succinimidyl propionate. Following reaction, the PEG-nucleic acid conjugate is purified by gel electrophoresis or liquid chromatography to separate fully-, partially-, and un-conjugated species. Multiple PAG molecules concatenated (e.g., as random or block copolymers) or smaller PAG chains can be linked to achieve various lengths (or molecular weights). Non-PAG linkers can be used between PAG chains of varying lengths.

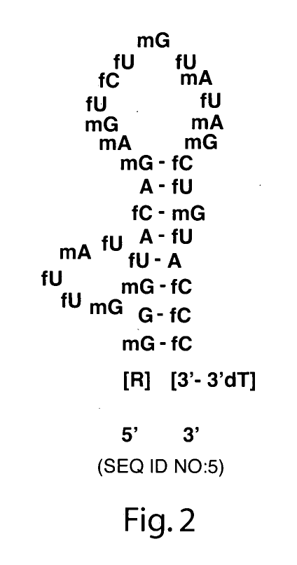

PAG-derivatization of a reactive nucleic acid. High molecular weight PAG-nucleic acid-PAG conjugates can be prepared by reaction of a mono-functional activated PEG with a nucleic acid containing more than one reactive site. In one embodiment, the nucleic acid is bi-reactive, or bi-activated, and contains two reactive sites: a 5′-amino group and a 3′-amino group introduced into the oligonucleotide through conventional phosphoramidite synthesis, for example: 3′-5′-di-PEGylation as illustrated in FIG. 2. In alternative embodiments, reactive sites can be introduced at internal positions, using for example, the 5-position of pyrimidines, the 8-position of purines, or the 2′-position of ribose as sites for attachment of primary amines. In such embodiments, the nucleic acid can have several activated or reactive sites and is said to be multiply activated. Following synthesis and purification, the modified oligonucleotide is combined with the mono-activated PEG under conditions that promote selective reaction with the oligonucleotide reactive sites while minimizing spontaneous hydrolysis. In the preferred embodiment, monomethoxy-PEG is activated with succinimidyl propionate and the coupled reaction is carried out at pH 8.3. To drive synthesis of the bi-substituted PEG, stoichiometric excess PEG is provided relative to the oligonucleotide. Following reaction, the PEG-nucleic acid conjugate is purified by gel electrophoresis or liquid chromatography to separate fully-, partially-, and un-conjugated species. FIG. 16 illustrates two strategies for synthesizing PEGylated nucleic acid aptamers.

The linking domains can also have one ore more polyalkylene glycol moieties attached thereto. Such PAGs can be of varying lengths and may be used in appropriate combinations to achieve the desired molecular weight of the composition.

Pharmaceutical Compositions

The invention also includes pharmaceutical compositions containing aptamer molecules. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat or prevent a pathology, such as a disease or disorder, or alleviate the symptoms of such disease or disorder in a patient. For example, compositions of the invention can be used to treat or prevent a pathology associated with inflammation. Compositions of the invention are useful for administration to a subject suffering from, or predisposed to, a disease or disorder which is related to or derived from a target to which the aptamers specifically bind.

For example, the target is a protein involved with inflammation, for example, the target protein causes or contributes to inflammation.

Compositions of the invention can be used in a method for treating a patient having a pathology. The method involves administering to the patient a composition comprising aptamers that bind a target (e.g., a protein) involved with the pathology, so that binding of the composition to the target alters the biological function of the target, thereby treating the pathology.

The patient having a pathology, e.g. the patient treated by the methods of this invention can be a mammal, or more particularly, a human.

In practice, the compounds or their pharmaceutically acceptable salts, are administered in amounts which will be sufficient to exert their desired biological activity.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixirs, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 7500 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Infused dosages, intranasal dosages and transdermal dosages will range between 0.05 to 7500 mg/day. Subcutaneous, intravenous and intraperitoineal dosages will range between 0.05 to 3800 mg/day.

Effective plasma levels of the compounds of the present invention range from 0.002 mg/mL to 50 mg/mL.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### Pharmacokinetic and Biodistribution Modulation of Aptamers Via Conjugation

Aptamer synthesis. The nucleotide sequence and predicted secondary structure of the parent oligonucleotide for the TGFb aptamer ARC83(SEQ ID NO 5) is shown in FIG. 2 (Pagratis, et al. (2002), High affinity TGFb nucleic acid ligands and inhibitors. USA, Gilead Sciences, Inc). Syntheses of ARC83 and the corresponding fully 2′-O-Me modified variant, ARC159 (SEQ ID NO 15), were performed using standard solid-phase phosphoramidite chemistry, followed by ion-exchange high pressure liquid chromatography (HPLC) or polyacrylamide gel electrophoresis (PAGE) purification. The ARC83 aptamer was synthesized by Dharmacon, Inc., Lafayette, Colo.

Synthesis of Aptamer Conjugates.

ARC83 NH₂— mGGmGmGfUfUmAfLfUAfCAmGmAmGfUfCfUmGfUmAfUmAmGfCfUmGfUAfCfCfC-3T (SEQ ID NO 5) was synthesized using standard procedures. The terminal amine function was attached to the aptamer nucleotide sequence using a six carbon linker as depicted below:

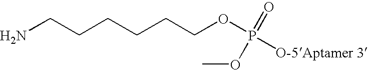

ARC83 (SEQ ID NO 5) was conjugated to different modifying moieties to generate the following conjugates: 20 kDa PEG (ARC120); 40 kDa PEG (ARC122); HIV Tat peptide (ARC156); antennapedia (Ant)-derived peptide (ARC157); or poly-arginine (Arg7) (ARC 158). The various modifications to ARC83 were made post-synthetically via amine-reactive chemistries. For the PEG conjugates, ARC83 was dissolved in 100 mM sodium carbonate buffer, pH 8.5 to a concentration of 1 mM, and was reacted for 1 hr with a 2.5 molar excess of mPEG-SPA (MW 20 kDa) or mPEG2-NHS ester (MW 40 kDa) (Shearwater Corp., Huntsville, Ala.) in an equal volume of acetonitrile. The resulting products were then purified by reverse phase HPLC on Hamilton PRP-3 columns with acetonitrile, 50 mM TEAA as an eluent.

The peptide conjugates were prepared by reacting pyridyldithio-activated aptamers with C-terminal cysteine containing peptides. For this procedure, ARC83 was dissolved to 2 mM in 100 mM sodium carbonate buffer, pH 8.5, and a 6-fold molar excess of N-succinimidyl 3-(2-pyridyldithio) proprionate (SPDP, Pierce, Rockford, Ill.) was added. After 90 min at room temperature the reactive intermediate was recovered through precipitation with isopropanol, resuspended in PBS buffer pH 7.4, and reacted for 3 hrs at room temperature with a 2 mM solution of RKKRRQRRRPPQC (Tat) (SEQ ID NO 6), RQIKIWFQNRRMKWKKGGC (Ant) (SEQ ID NO 7) or RRRRRRRC (Arg7) (SEQ ID NO 8) (Tufts University Core Facility, Boston, Mass.) in an equal volume of formamide. Products were purified by electrophoresis on preparative 10 % (w/v) polyacrylamide gels. A 5′-cholesterol-modified oligonucleotide (ARC155, SEQ ID NO 11) was also prepared by standard automated solid-phase synthesis using a cholesteryl-TEG phosphoramidite (Glen Research, Sterling, Va.).

For each of the individual sequences listed in Table I below, all 2′-OMe modifications are indicated by an “m” preceding the corresponding nucleotide; all 2′-fluoro modifications are indicated by an “f′ preceding the corresponding nucleotide; and any purine or pyrimidine appearing without an “m” or “f′ preceding the nucleotide indicates a 2′-OH residue; a “PEG20K” represents a conjugated 20 kDa polyethylene glycol; a “PEG40K” represents a conjugated 40 kDa polyethylene glycol; a “C” represents a conjugated cholesterol; “tat” represents a conjugated tat peptide; “ant” represents a conjugated ant peptide; “arg” represents a conjugated arg₇ peptide; NH₂ represents a 5′ hexylamine modification; NH represents a hexylamine linker and 3T denotes an inverted 3′-3′ deoxythymidine cap at the 3′ terminus.

TABLE 1
Aptamer Compositions

| | | |
|---|---|---|
| ARC# | SEQ ID No. | Sequence (5′ → 3′) |

| | | |
|---|---|---|
| ARC83 | 5 | NH₂- mGGmGmGfUfUmAfUfUAfCAmGmAmGfUfCfUmGfUmAfUmAmGfCfUmGfUAfCfCfC-3T |
| ARC120 | 9 | PEG20K-NH-mGGmGmGfUfUmAfUfUAfCAmGmAmGfUfCfUmGfUmAfUmAmGfCfUmGfUAfCfCfC-3T |
| ARC122 | 10 | PEG40K-NH-mGGmGmGfUfUmAfUfUAfCAmGmAmGfUfCfUmGfUmAmGfCfUmGfUAfCfCfC-3T |
| ARC155 | 11 | C-NH-mGGmGmGfUfUmAfUfUAfCAmGmAmGfUfCfUmGfUmAfUmAmGfCfUmGfUAfCfCfC-3T |
| ARC156 | 12 | tat--NH-mGGmGmGfUfUmAfUAfCAmGmAmGfUfCfUmGfUmAfUmAmGfCfUmGfUAfCfCfC-3T |
| ARC157 | 13 | ant--NH-mGGmGmGfUfUmAfUfUAfCAmGmAmGfUfCfUmGfUmAfUmAmGfCUmGfUAfCfCfC-3T |
| ARC158 | 14 | arg₇-NH-mGGmGmGfUfUmAfUfUAfCAmGmAmGfUfCfUmGfUmAfUmAmGfCfUmGfUAfCfCfC-3T |
| ARC159 | 15 | NH₂-mGmGmAmGmGmUmUmAmUmUmAmCmAmGmAmGmUmCmUmGmUmAmUmAmGmCm- |
| | | UmGmUmAmCmUmCmC-3t |

Aptamer conjugate composition was verified by polyacrylamide and capillary gel electrophoresis, HPLC, and/or matrix-assisted laser desorption ionization time of flight mass spectrometry (MALDI-TOF). Conjugates were formulated for dosing after precipitation and desalting using CENTRISEP™ filters (Princeton Separations Inc., Adelphia, N.J.).

[³H]-labeling of aptamers. ARC83 and ARC159 were tritiated (ViTrax, Inc. Placentia, Calif.) using a heat-catalyzed tritium exchange reaction (Graham, et al. (1993), Nucleic Acids Res. 21(16): 3737-43). ARC83 and ARC159 reaction products were determined to have high radiochemical purity (>98 %) and specific activities of 850 μCi/mg and 760 μCi/mg, respectively. Tritium did not back-exchange from [³H]-ARC83 or [³H]-ARC159 at temperatures below 95° C. and the radiolabel was considered to have a physical stability suitable for in vivo experimental conditions. Samples of the tritiated 20 kDa PEG conjugate (ARC120) and the Arg₇-oligonucleotide (ARC158) were prepared as described above on a small scale, using [³H]-ARC83 as starting material, followed by PAGE purification.

Dosing formulations. Aptamers for the pharmacokinetic study were dissolved in 1× phosphate-buffered saline (PBS) (137 mM NaCl, 2.7 mM KCl, 10 mM NaHPO₄, 2 mM KH₂PO₄, pH 7.2) at a concentration of 1 mg/ml prior to administration. For the biodistribution experiments involving tritiated aptamers, dosing formulations were prepared in 1×PBS to a final concentration of 1 mg/ml, with specific activities of 25 μCi/mg (ARC83), 52 μCi/mg (ARC120), 37 μCi/mg (ARC158) and 37 μCi/mg (ARC159).

Animals and experimental procedures. Pharmacokinetic and biodistribution studies were performed using 8-10 week-old male Sprague-Dawley rats (250-300 g, Charles River Laboratories, Wilmington, Mass.). Rats were fed ad libitum with a standard laboratory diet, housed and kept under controlled conditions (12 hr light cycle, 20° C.) prior to experimentation. For experiments in which urine was collected, animals were housed in metabolic cage units with free access to food and water. Prior to dosing, rats were divided into treatment groups and each received a single intravenous administration of aptamer at a dose of 1 mg/kg body weight. For some experiments, animals were fitted with femoral and jugular vein catheters (Hilltop Lab Animals, Scottsdale, Pa.) and doses, at a volume of 1 ml/kg, were administered as a bolus injection into the femoral vein catheter. Immediately following intravenous administration, the dosing catheter was flushed with 0.5 ml of 0.9% saline. Blood samples were collected from replicate animals (n=3) in each group at time points of −0.25 (i.e., pre-dose), 0.25, 1, 3, 12, and 48 hrs via the jugular vein catheter. At 48 hrs, all animals were euthanized, and selected tissues were collected. For biodistribution studies involving tritiated aptamers, each animal received a single 1 mg/kg intravenous bolus dose of [³H]-labeled aptamer per animal: ARC83, 6.6 μCi; ARC120, 13.9 μCi; ARC158, 9.6 μCi; ARC159, 9.7 μCi, respectively. At time points of 3, 12, and 24 hours post-dosing, subgroups of animals (n=2 per time point) for each aptamer conjugate were euthanized, and blood samples and selected tissues were collected. Blood samples were centrifuged immediately for isolation of plasma, as described below. For the animals that were euthanized at 24 hours post-dosing, urine was collected over the following intervals: 0-3, 3-6, 6-12, and 12-24 hours post-dosing (n=2 animals per group). For radioactive biodistribution studies involving tritium, control animals (n=1 rat per time point) were dosed in parallel with cold, i.e., unlabeled, versions of the same aptamers or aptamer conjugates, and blood, tissue, and urine samples were collected, as described above.

Biological sample collection and processing. Blood samples (0.4 ml) collected from animals at specified time points were drawn into tubes containing sodium-EDTA-containing as an anticoagulant (1.8 ml vacutainers., BD Biosciences, San Jose, Calif.). Blood samples were placed immediately on wet ice and then processed by centrifugation for 10 min at approximately 4° C. to yield plasma. Plasma samples were stored at −20° C. until analysis. In biodistribution studies involving non-radioactive aptamers, selected tissues, e.g., brain, heart, kidneys, liver, spleen, and bone marrow from both femurs, were collected at 48 hrs. The brain, heart, kidneys, liver and spleen were rinsed with 0.9% saline and then blotted dry prior to weighing. Organs were homogenized in saline using a polytron homogenizer to produce a 50% tissue homogenate [1:1 (w/w) tissue:saline]. Each bone marrow sample, from both femurs combined, was homogenized in saline to produce a 20% tissue homogenate [1:4 (w/w) tissue:saline]. All tissue homogenates were stored at −20° C. prior to analysis. To determine biodistribution of [³H]-labeled aptamers, selected tissues, e.g., liver, kidneys, lungs, heart, spleen, brain, bone marrow (both femurs), gastrointestinal tract, eyes, and mediastinal lymph nodes, were harvested at 3, 12 and 24 hrs post-administration.

### Example 2

### Hybridization-Based Dual-Capture Assay for Aptamer Quantitation

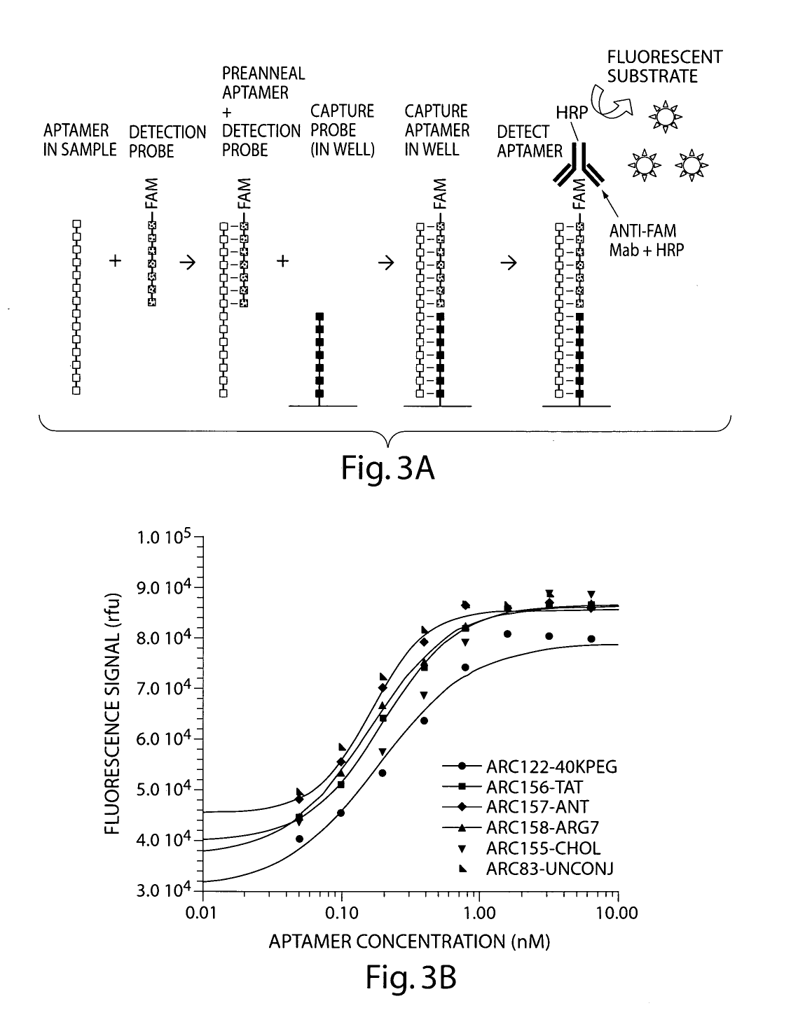

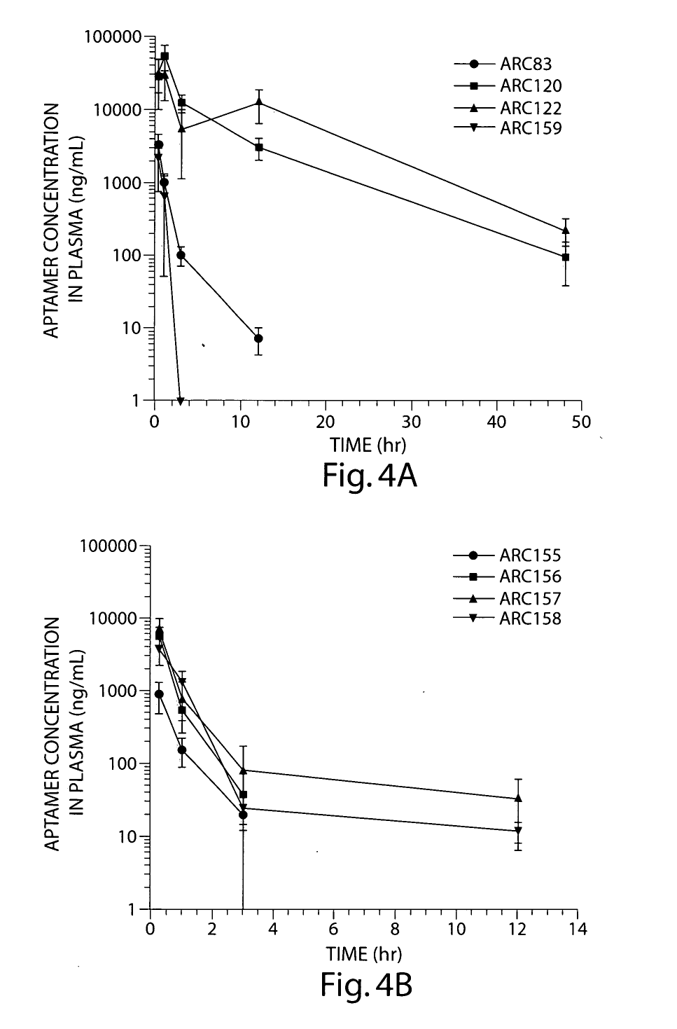

To facilitate in vivo studies, a hybridization-based dual probe capture assay with enzyme-linked fluorescent readout for monitoring the concentration of intact, undegraded aptamer in biological samples was developed (shown schematically in FIG. 3A). Generally, the assay used a capture probe attached to a solid support (e.g., a 96-well plate bottom), and a FAM-labeled detection probe. When the aptamer-containing sample and probes are combined in the assay well, the pre-immobilized capture probe forms a hybrid with the 5′ end of the oligonucleotide (e.g., aptamer) to be detected and the pre-annealed detection probe formed a hybrid with the 3′ end. Following extensive washing to remove free probe molecules, an anti-FAM-HRP conjugate was combined to generate a fluorescent signal proportional to the concentration of retained probe-aptamer complex.

This hybridization-based dual-capture pseudo-ELISA (FIG. 3A) was the primary analytical method used to measure the concentration of intact, nonradioactive aptamer in plasma and tissue homogenate samples. In this assay, a biotinylated capture probe (ARC179, SEQ ID NO 16, 5′ ACUCUGUAAUAACCCC-[spacer18]-biotin) was pre-immobilized in the wells of a 96-well microplate at a binding solution concentration of 125 nM (×100 ul/well=12.5 pmole/well) for 3 hrs. As used herein, the term “spacer18” refers to a PEG-6 (i.e., 6 ethylene unit, 18 carbon atoms) linker that is 18 atoms long. The plate wells were washed 5 times using a Biotek Elx405 plate washer with 1× Dulbecco's PBS. The plates were then blocked with 150 ul/well of a solution containing 1×PBS, 0.05% Tween-20, 0.025% tRNA. Plates were washed again, covered, and stored at 4° C. until use. In separate tubes, the samples were annealed in a buffer containing the FAM-labeled detection probe (ARC180, SEQ ID No.17, 5′FAM-[spacer18]-GGGUACAGCUAUACAG, at 200 nM) at 90° C. for 10 min, then quenched on ice. Standard control samples and the pre-annealed plasma/tissue sample-detection probe solutions were then pipetted (typically 1:5-1:1000-fold dilutions of the sample were assayed) into the plate wells containing the immobilized biotin capture probe, and annealed at 45° C. for 2.5 hrs. Plates were then washed again, and filled with 100 ul/well of a solution containing 1 ug/ml of anti-fluorescein monoclonal antibody conjugated to horse radish peroxidase (anti-FITC MAb-HRP, Molecular Probes, Eugene, Oreg.) in 1×PBS, and incubated for 1.3 hrs. Plates were washed again as described above. Wells were then filled with 100 ul of a solution containing a fluorogenic HRP substrate (QUANTABLU™ Pierce Chemical, Rockford, Ill.), and incubated for 30-45 minutes, protected from light. After incubation, 100 ul/well of a stop solution was added to quench the fluorescent precipitate-producing reaction. Plates were read immediately on fluorescence microplate reader (Fusion, Packard Biosciences, Billerica, Mass.) with fluorescence excitation at 335-350 nm and emission detected at 460 nm. Each well was read 10 times, with a 1 min interval between plate reads. The mean relative fluorescence value (RFU), standard deviation, and % CV for all reads of each plate were written to an electronic file for subsequent analysis.

Data files were analyzed using KALEIDAGRAPH™ to fit the RFU signal vs. concentration curves for duplicate or triplicate control standards included on each plate. Once a standard concentration curve was generated for a given sample plate, the plasma/tissue sample well signals were analyzed to determine the concentration of full-length aptamer present in each sample.

Measurement of radioactivity. Each sample was prepared for analysis of total [³H]-radioactivity in duplicate. Aliquots of known volume of each urine, metabolic cage rinse, and plasma sample were mixed with 10 ml of ULTIMA GOLD™ (Packard BioSciences Co., Meriden, Conn.) liquid scintillation cocktail for direct analysis by liquid scintillation counting (LSC). All other samples (or aliquots of samples) were combusted prior to LSC analysis. Aliquots of samples analyzed by combustion were weighed into combustion boats and combusted in a Harvey Biological Materials Oxidizer (Model OX500 or OX300, R. J. Harvey Instrument Co., Hillsdale, N.J.). Each aliquot was combusted for 4 min. The liberated [³H]₂O was trapped in 15 ml of Monophase S liquid scintillation cocktail (Packard Bioscience Co.). A solution of each radiolabeled aptamer was used as an oxidation standard. The LSC results for combusted samples were corrected for the efficiency of the oxidizer as determined on the day the specific samples were combusted.

Radioactivity was quantitated by LSC using a Beckman Model LS 6000TA or LS 6500 liquid scintillation spectrophotometer (Beckman Instruments Inc., Fullerton, Calif.). Count data were automatically corrected for chemical quench, as determined using a ¹³⁷Cs external standard. All samples were counted for 10 minutes or until a 2-σ error of 1% was achieved. Analyses were considered acceptable if the duplicate dpm/g or dpm/ml values were within 10% of the mean value, provided the mean per aliquot analyzed was >100 dpm. The sensitivity of the radio-analytical procedures was estimated assuming that a minimum of 150 dpm (above a background of approximately 50 dpm) per aliquot assayed was required for quantitation. Approximate mean sample size, aliquot size, and dose were used in the calculations. The [³H]-content of each sample was adjusted for total tissue weight and expressed as a percentage of the administered dose, or as a the equivalent concentration of aptamer (μg equiv/ml or μg equiv/gram). In terms of concentration, actual quantitation limits ranged from 0.0013 μg/g to 0.26 μg/g. In terms of percent of dose, actual quantitation limits ranged from 0.00051% of dose to 0.025% of dose.

Pharmacokinetic models and parameters. An initial pharmacokinetic analysis of the scrambled anti-TGFβ2 aptamer (ARC83) in rats following bolus intravenous injection was carried out by Gilead Sciences, Inc. (Pagratis, Lochrie et al. (2002)). The observed plasma pharmacokinetic (PK) profile of the anti-TGFβ2 aptamer appeared to be biphasic and described by a two-compartment model. The pharmacokinetic profile of the anti-TGFβ2-derived aptamer (ARC83) was also well-described by a two-compartment model. It should be noted, however, that the behavior of the unconjugated, fully 2′-O-Me version of the aptamer (ARC159) showed a monophasic profile and could only be modeled by a one-compartment model or by noncompartmental analysis. The concentration vs. time data for all aptamer conjugates was imported into software package WINNONLIN™ (Pharsight Corp., Mountainview, Calif.) for analysis.

### Example 3

### Detection of Intact Aptamers From Biological Samples

The primary analytical method used to measure the concentration of intact, nonradioactive aptamers in biological samples, e.g., plasma, tissue and urine, was the hybridization-based dual-capture pseudo-ELISA described above (See FIG. 3A). Additional bioanalytical methods used included capillary gel electrophoresis (CGE) and MALDI-TOF. For CGE analysis, samples spiked with 50 pmole of an oligonucleotide (T₂₀) internal standard were incubated in buffer (60 mM Tris-Cl, pH 8.0, 100 mM EDTA, 0.5% SDS) containing proteinase K at 500 μg/ml at 65° C. for 4 hrs. Digests were extracted twice with phenol/chloroform, and then precipitated with ammonium acetate. CGE (Beckman P/ACE 5010) was performed at 25° C. using 10% polyacrylamide gel-filled capillaries (20 cm) and an applied voltage of 550 V/cm. Elution of oligonucleotides from the gel was monitored using UV detection at 260 nm. Under these conditions, resolution of aptamers from chain-shortened metabolites (i.e., oligonucleotide biodegradation products, wherein N nucleotides (N=1, 2 or 3) are removed from the 3′-end of the molecule) was achieved. MALDI-TOF analysis was performed using an ABI Voyager-DE PRO mass spectrometer in linear mode (Applied Biosystems, Foster City, Calif.). Urine samples for mass spectrometry were purified on ZIPTIP™ C18 pipette tips (Millipore, Billerica, Mass.).

### Example 4

### Plasma Pharmacokinetic and Tissue Distribution Properties of Stabilized Aptamer

The plasma pharmacokinetic and tissue distribution properties of stabilized aptamer compositions were defined and the ability to alter both plasma half-life and biodistribution through use of small and high molecular weight polymer tags was assessed. Oligonucleotide ARC83 (FIG. 1) is 32 nucleotides long and is derived from a previously described aptamer specific for TGFβ-2 (Pagratis et al., 2002). ARC83 contains internal 2′-F and 2′-O-Me modifications, as well as a 3′ inverted-dT cap for enhanced stability against nuclease attack. The 5′-terminal amino modifier is useful for subsequent conjugation to amine-reactive moieties, such as the various modifying moieties. Though identical in terms of nucleotide sequence to the reported anti-TGFβ-2 aptamer, ARC83 is an inactive variant which does not bind TGFβ-2 due to scrambling of selected positions in the molecule with regard to 2′ sugar modification. ARC159 is also a biologically inert variant of ARC83 where every 2′ position bears an O-Me substitution. These inactive aptamer versions were used in an effort to eliminate any potential experimental error that could be caused by an aptamer-target interaction, as such experimental error could alter the pharmacokinetic and biodistribution measurements for each aptamer. Both ARC83 and ARC159 exhibit a high degree of resistance to plasma nucleases in vitro, as determined by denaturing PAGE following incubation of radiolabeled aptamers in the presence of 95% rat plasma. The in vitro half-life of ARC83 is approximately >48 hrs, while the fully 2′-O-Me composition aptamer, ARC159, is almost entirely stable up to 96 hrs.

As shown in FIG. 3B, the lower limit of detection achievable with the hybridization-based dual probe capture assay was 50-100 pM (˜0.8 ng/ml, based on aptamer weight without conjugation), while the upper limit of detection was ˜25 nM (264 ng/ml, based on aptamer weight without conjugation). The resulting dynamic range was ≧250-fold. To ensure that sample concentrations fell within the dynamic range for the assay, a range of dilutions of each sample was analyzed. For time points shortly after the initial dosing, larger dilution factors (-1 :100-1:1000) were used, whereas for later time points, dilution factors of 1:5-1:10 were necessary for accurate detection and quantitation. In general, several sample dilution points were within the linear dynamic range of the assay, and thus, provided a measure of the precision of the assay. The data sets resulting from the dual-capture ELISA assay constitute concentration vs. time data for the aptamer conjugates. This data was then fitted, analyzed, and compared against data generated by pharmacokinetic models of the system under investigation, in order to extract parameters such as the characteristic half-life of the aptamer in the blood, the maximum concentration observed, the total integrated dose administered, overall clearance rate, and volume of distribution.

### Example 5

### Pharmacokinetic Profiles of Aptamer Conjugates in Plasma

Using the hybridization-based assay, the concentration of aptamer-derived conjugates expressed as a function of time following intravenous bolus administration to rats was determined. Aptamer conjugates were formed by attachment of activated tags to a synthetically incorporated 5′-amino group of ARC83 as described in Example 1. Conjugates that were tested included 20 kDa and 40 kDa PEGs, cholesterol, and a set of peptides previously reported to facilitate extravasation and/or cellular uptake of conjugated molecules, including an Antennapedia-derived peptide, a Tat-derived peptide, and Arg₇ (Antopolsky, et al. (1999); Zubin, et al. (1999); Astriab-Fisher, et al. (2000); Lebedeva, et al. (2000); Manoharan (2002)). The standard curves for a variety of different aptamer conjugates are shown in FIG. 3B. The limit of detection for the assay is relatively unaltered as a result of aptamer conjugation. The presence of plasma or a variety of tissue homogenates decreased the sensitivity of the assay under typical dilution conditions to a lower limit of detection of approximately 100-200 pM.

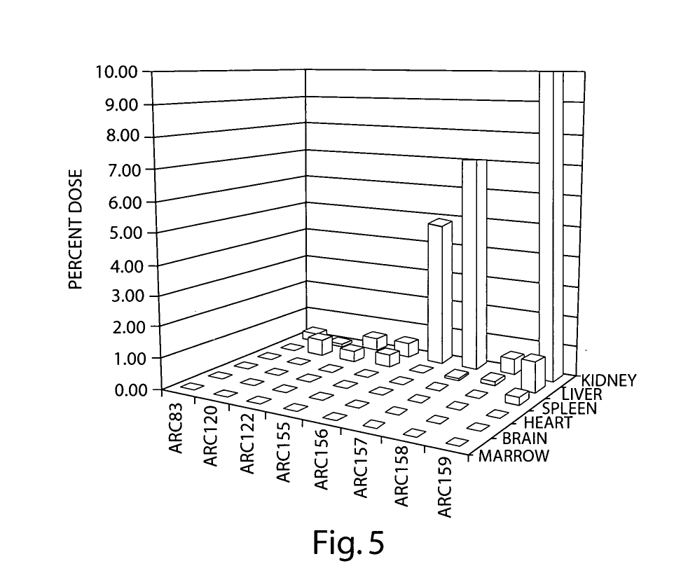

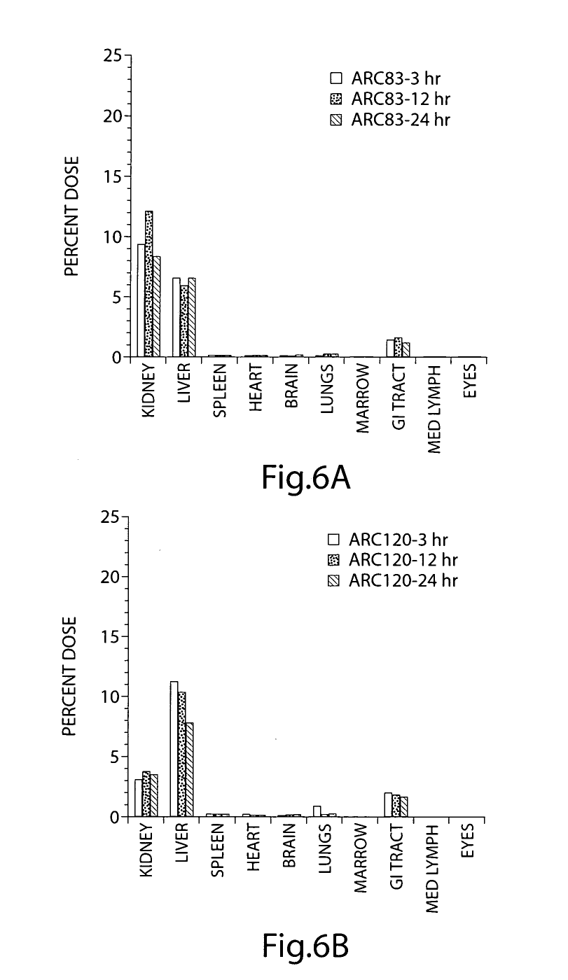

FIG. 4 depicts the in vivo plasma pharmacokinetic profiles in rat for unconjugated and conjugated aptamers measured at intervals over 48 hrs. Primary and secondary pharmacokinetic parameters extracted from analysis of the concentration vs. time data in the context of a biphasic, two-compartment model are presented in Tables 2A and 2B. The listed concentrations for both plasma and tissue analysis refer only to full-length aptamer sequence, and do not include the molecular weight of an associated modifying moiety (such as PEGylation). The unconjugated 2′-F/2′-O-Me-modified aptamer (ARC83) was reasonably long-lived, displaying a t_{1/2} (β) of nearly 5 hrs and a mean residence time of approximately 1.7 hr. The relatively large volume of distribution (approximately 460 ml/kg) of ARC83 suggested that, even in the absence of conjugation, the aptamer does distribute to tissues to some degree (FIG. 4A and Tables 2A and 2B). As expected, the 40 kDa PEG-conjugate (ARC122) showed a substantially longer half-life in circulation, t_{1/2} (β) of nearly 12 hrs, and a significantly reduced volume of distribution, 144 ml/kg. Notably, the mean residence time for the 40 kDa PEG-conjugate, nearly 16 hrs, was approximately 10-fold greater than that for unconjugated aptamer. Consistent with these results, the 20 kDa PEG-conjugate (ARC 120) exhibited intermediate values for half-life in circulation, t_{1/2} (β) of approximately 7 hr, and mean residence time, close to 8 hr, relative to both unconjugated aptamer and to the 40 kDa PEG conjugate (FIG. 4A and Tables 2A and 2B). While PEGylation slowed clearance of the aptamer from the circulatory volume, neither 20 kDa nor 40 kDa PEG conjugation appeared to enhance the distribution of the aptamer into extravascular spaces as judged by their calculated volumes of distribution (Vss, Table 2B). However, the 20 kDa PEG conjugate (ARC120) showed a significantly higher AUC value relative to the 40 kDa PEG conjugate (ARC122), suggesting that 20 kDa PEGylation may more effectively promote aptamer exposure to organs and tissues.

Tables 2A and 2B. Pharmacokinetic parameters of aptamer conjugates

TABLE 2A

| | Modifying | | | | |
|---|---|---|---|---|---|
| ARC # | Moiety | AUC ng*hr/mL | Cmax ng/mL | A ng/mL | B ng/mL |
| ARC83 | none | 3573.39 ± 262 | 5009.40 ± 381 | 4926.20 ± 360 | 83.20 ± 73 |
| ARC120 | 20K PEG | 146343.66 ± 3280 | 31323.00 ± 522 | 21711.83 ± 1153 | 9611.79 ± 1149 |
| ARC122 | 40K PEG | 110009.41 ± 70996 | 14223.19 ± 10348 | 8106.47 ± 9904 | 6116.72 ± 5603 |
| ARC155 | cholesterol | 778.58 ± 24 | 1711.05 ± 85 | 1649.00 ± 78 | 62.06 ± 26 |
| ARC156 | TAT | 4222.22 ± 381 | 12885.00 ± 1670 | 12855.40 ± 1661 | 30.40 ± 54 |
| ARC157 | ANT | 6211.96 ± 53 | 16229.75 ± 131 | 16120.14 ± 130 | 109.60 ± 6 |
| ARC158 | Arg₇ | 3983.08 ± 715 | 5634.51 ± 784 | 5623.24 ± 777 | 11.27 ± 40 |
| ARC159 | 2′-OMe | 2013.84 ± 180 | 3614.53 ± 464.56 | 3614.53 ± 464.56 | — |

TABLE 2B

| | Modifying | t_{1/2}(α) | t_{1/2}(β) | MRT | CL | Vss |
|---|---|---|---|---|---|---|
| ARC # | Moiety | hr | hr | hr | mL/kg/min | mL/kg |
| ARC83 | none | 0.42 ± 0.04 | 4.88 ± 3.47 | 1.66 ± 0.91 | 4.66 ± 0.34 | 464.28 ± 232.08 |
| ARC120 | 20K PEG | 1.47 ± 0.11 | 7.23 ± 0.57 | 7.81 ± 0.42 | 0.11 ± 0.003 | 53.34 ± 2.21 |
| ARC122 | 40K PEG | 0.60 ± 1.63 | 11.68 ± 11.77 | 15.84 ± 15.09 | 0.15 ± 0.09 | 144.97 ± 94.32 |
| ARC155 | cholesterol | 0.26 ± 0.02 | 1.79 ± 0.53 | 0.83 ± 0.14 | 21.40 ± 0.67 | 1067.20 ± 163.54 |
| ARC156 | TAT | 0.22 ± 0.02 | 4.93 ± 9.53 | 0.66 ± 0.96 | 3.95 ± 0.36 | 156.38 ± 219.07 |
| ARC157 | ANT | 0.22 ± 0.002 | 6.87 ± 0.45 | 1.99 ± 0.16 | 2.68 ± 0.02 | 320.94 ± 23.09 |
| ARC158 | Arg₇ | 0.46 ± 0.07 | 16.34 ± 60.77 | 2.19 ± 7.88 | 4.18 ± 0.75 | 549.65 ± 1907.22 |
| ARC159 | 2′-OMe | 0.39 ± 0.05 | — | 0.56 ± 0.07 | 8.28 ± 0.74 | 276.66 ± 35.59 |

The fully 2′-O-Me aptamer (ARC159, SEQ ID No. 15) displayed much more rapid elimination from plasma compared to ARC83. Indeed, ARC159 showed the shortest mean residence time (MRT) in the blood stream (approximately 30 min) of any of the aptamers tested (FIG. 4A and Table 3B). The pharmacokinetic profile of ARC159 was also distinctive in that it displayed a monophasic pharmacokinetic profile best described by one-compartment or noncompartmental models. In contrast to unconjugated aptamer, conjugates bearing cholesterol (ARC155) and Tat (ARC156) could not be detected in plasma by 12 hrs using the hybridization assay (FIG. 4B), which suggests an increase in their rates of clearance. The other cell permeating peptide-conjugates tested, ARC157 (Ant) and ARC158 (Arg₇) did not demonstrate marked differences from unconjugated aptamer (ARC83) in terms of their pharmacokinetic parameters (FIG. 4B and Tables 3A and 3B).

In a separate study, the levels of radiolabeled aptamers (including conjugates) in rats following intravenous bolus administration were detected and quantitated. Plasma concentrations of [³H]-aptamer equivalents for ARC83, ARC120, ARC158, and ARC159 over time showed the same trends seen for non-radioactively labeled aptamers measured using the hybridization-based assay. The relative abundance of full-length aptamer conjugates at all time points showed the following relation: [ARC120]>[ARC83]>[ARC158]>[ARC159], with concentrations of all aptamers decreasing from 3 to 24 hrs. The concentration of [³H]-ARC83 and [³H]-ARC158 equivalents were in the range of 450 to 700 ng/ml, whereas the concentration of [³H]-ARC159 equivalents ranged from approximately 200 to 400 ng/ml by 24 hrs after dosing. The concentrations for ARC120 (20 kDa PEG) [³H]-aptamer equivalent at 3 hours post-dosing were approximately an order of magnitude greater than the concentrations 3 hrs post-dosing for the other aptamers; the concentration of ARC120 subsequently decreased to a level similar to that for the other aptamers.

Plasma concentrations of unlabeled ARC83, ARC120, ARC158, and ARC159, as determined for animals dosed in parallel with cold aptamers by dual-capture ELISA were, 100-350, 250-2000, 20-50, and 15-50 ng/ml, respectively (FIGS. 4A and 4B). Taking the ratio of aptamer concentrations measured using the dual-hybridization ELISA assay to the aptamer concentrations obtained using liquid scintillation counting (LSC) provides an estimate of the fraction of full-length aptamer present in the plasma at each time point. The full-length fractions derived using this method range from 22-42% at 3 hrs for all aptamer conjugates tested.

### Example 6

### Analysis of Aptamer Distribution to Tissues

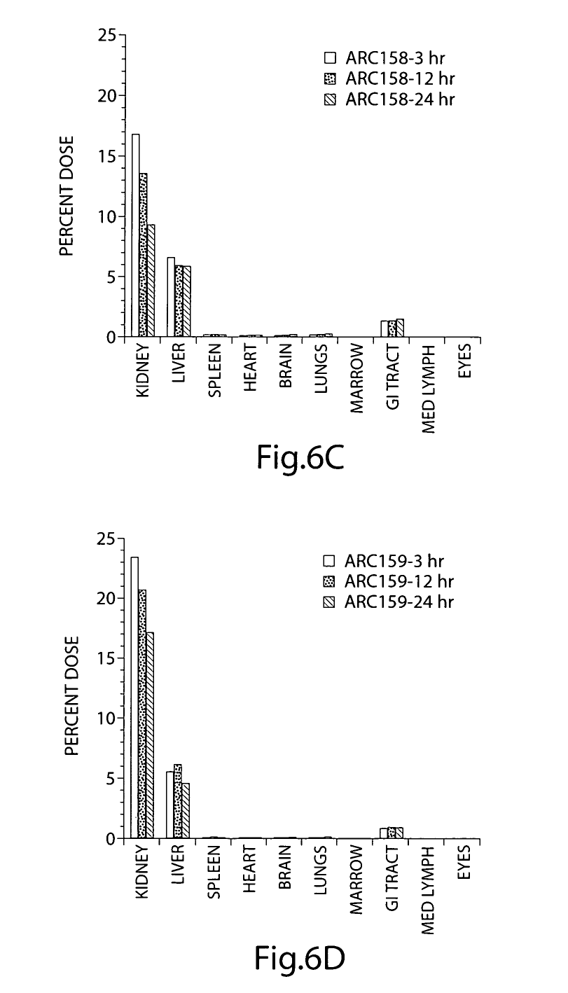

Aptamer concentrations in terminal tissue samples harvested 48 hrs. post-administration of the aptamer conjugate were quantified by dual capture ELISA. Significant levels of several aptamer species, most notably ARC159, were detected in kidney, and to a lesser extent, in liver and spleen (FIG. 5). While not intending to be bound by theory, the high levels of ARC159 found in these organs at 48 hrs relative to other aptamers may reflect the extremely high degree of intrinsic stability against nuclease attack in vivo conferred by saturating 2′-O-Me modification. The kidneys also represented the major target for distribution of the Tat (ARC156) and Ant (ARC157) peptide conjugates (FIG. 5).

Biodistribution of radiolabeled aptamers. Distribution of tritiated aptamers ARC83 (unconjugated), ARC120 (20 kDa PEG), ARC158 (Arg₇), and ARC159 (fully 2′-O-Me) to ten different organs or tissues, e.g., liver, kidney, lungs, heart, spleen, brain, bone marrow, gastrointestinal tract, eyes, and mediastinal lymph nodes, was determined in vivo over time in rats receiving a single intravenous bolus administration of each aptamer. Tissues, plasma, and urine were collected at intervals and analyzed for total radioactivity [³H] by oxidation and subsequent liquid scintillation analysis.

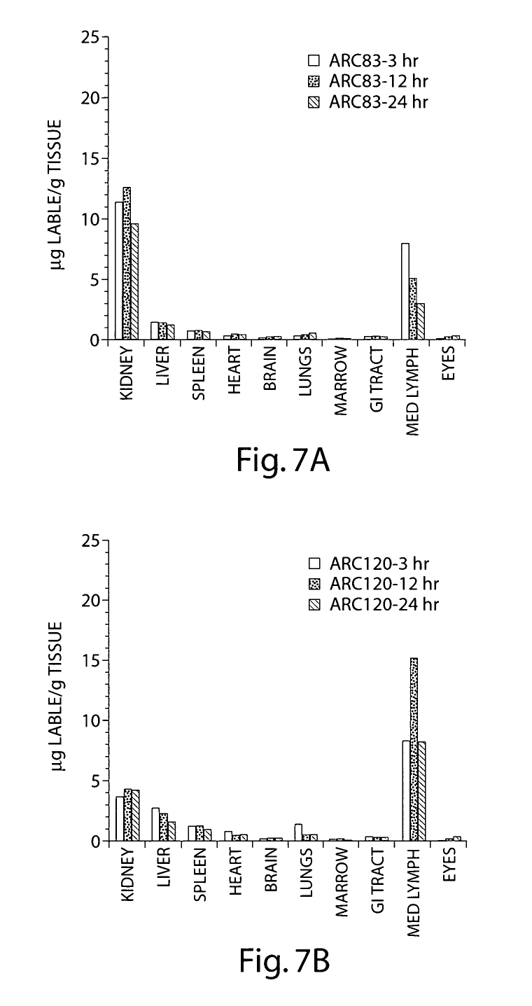

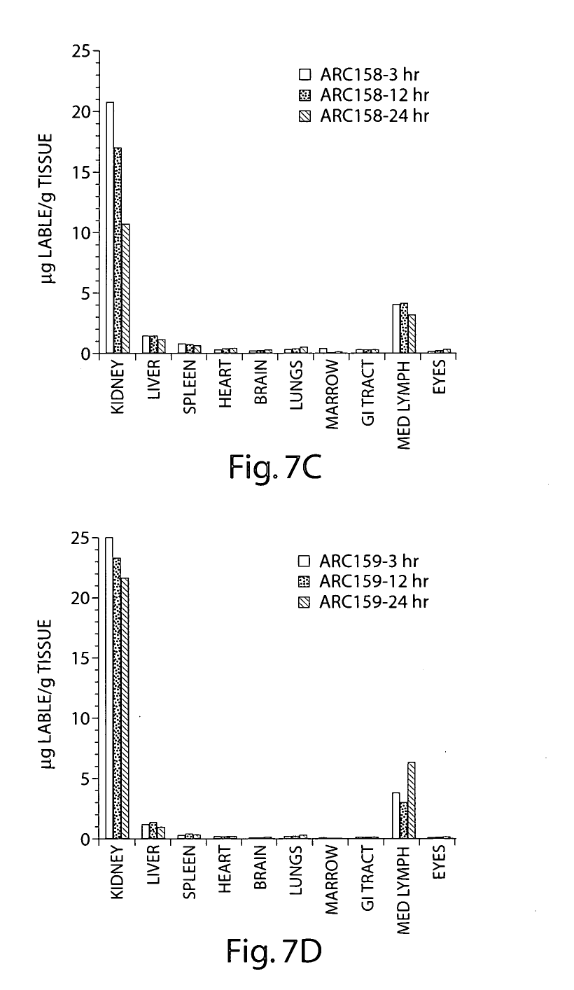

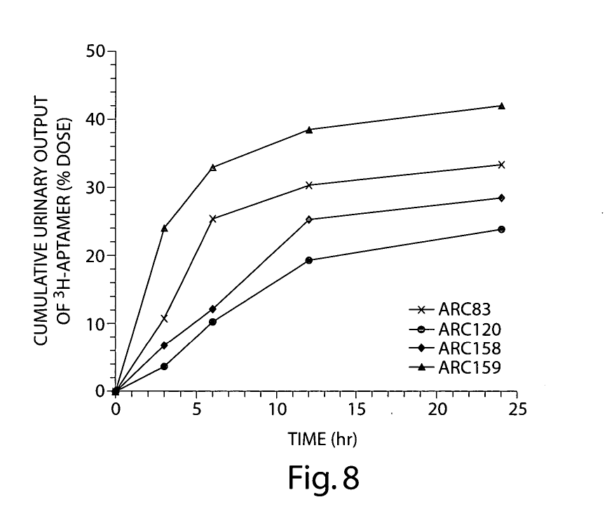

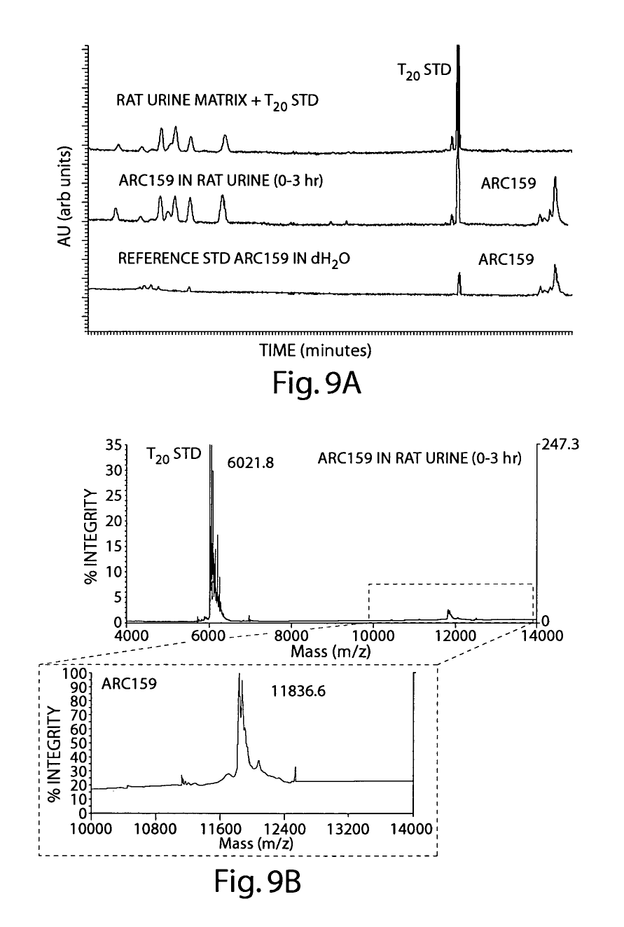

Among organs examined, the greatest uptake of [³H]-aptamer equivalents (normalized with respect to the administered dose) were found in the kidney and liver (FIGS. 6A-6D). The gastrointestinal tract also showed measurable levels of all aptamers. Qualitatively, aptamer biodistribution profiles established by 3 hrs were largely maintained over the duration of the 24 hr experiment. While the observed tissue distribution profiles of [³H]-aptamers were generally similar among the aptamers tested, there were a few notable differences. For [³H]-ARC120 (20 kDa PEG) (FIG. 6B), uptake by the kidneys accounted for <5% of the administered dose at all sampling times, whereas for ARC159 (fully 2′-O-Me) (FIG. 6D), uptake by the kidneys accounted for >20% of the administered dose. Indeed, levels of ARC 120 in the kidney were significantly below levels of all of the other aptamers at all time points examined, consistent with the ability of 20 kDa PEG conjugation to reduce renal filtration and thereby prolong aptamer residence in circulation. Conversely, except for the kidneys, dose-normalized levels of [³H]-ARC120 equivalents in the liver were higher than for the other aptamers (FIGS. 6A-6D). Overall, the subset of organs/tissues assayed accounted for uptake of ˜35-70% of the administered dose.

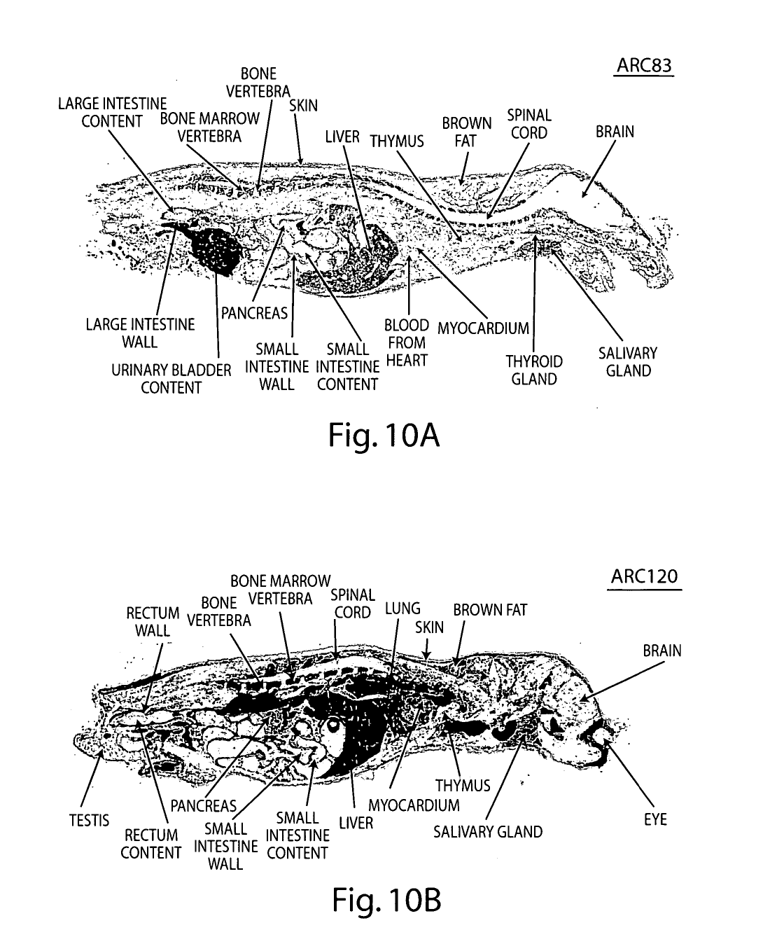

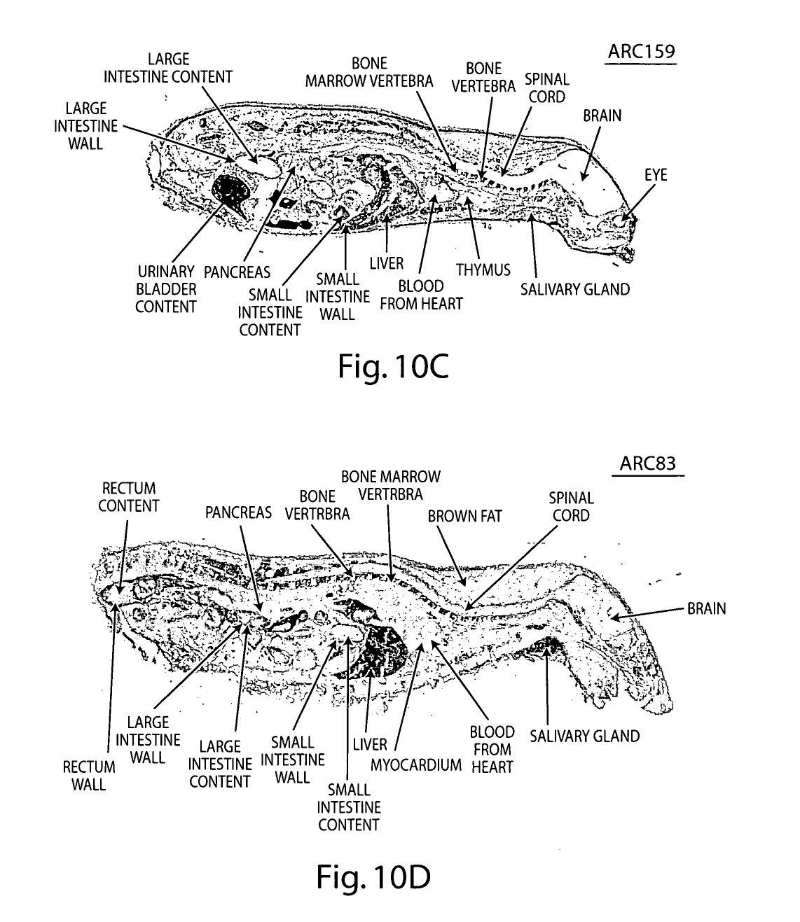

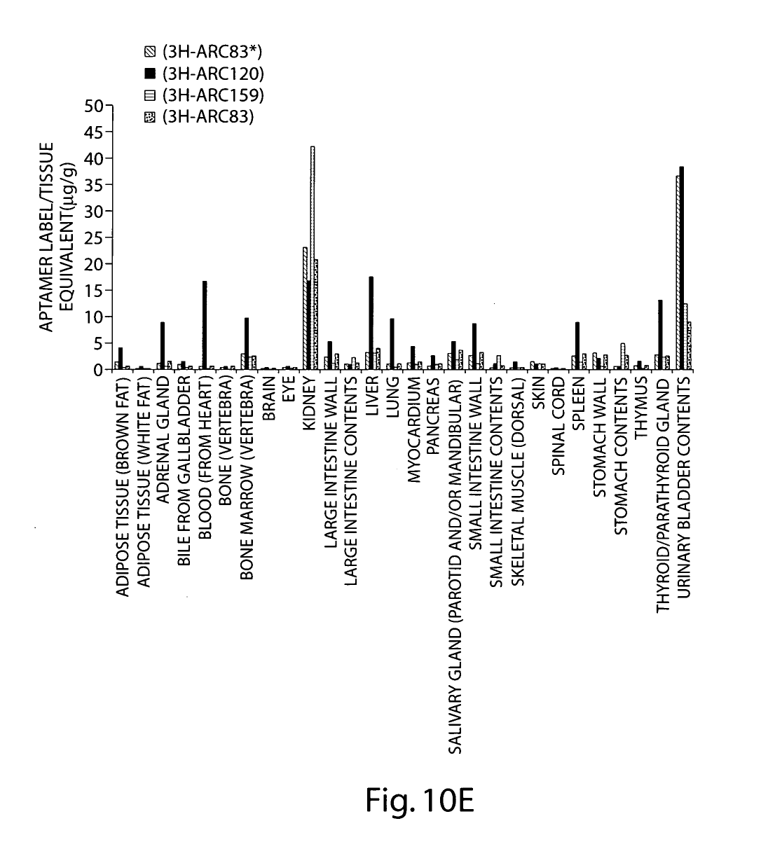

FIGS. 7A-7D show the distribution of [³H]-aptamer equivalents with data correction for organ or tissue weight. In general, greatest mass-normalized concentrations of [³H]-aptamer equivalents were seen in highly perfused organs such as kidney, liver, spleen, heart, and lungs. Comparatively high levels of each of the four aptamers or aptamer conjugates were also detected in the mediastinal lymph nodes (FIGS. 7A-7D). For animals dosed with [³H]-ARC83 (unconjugated) (FIG. 7A), [³H]-ARC158 (Arg₇) (FIG. 7C), and [³H]-ARC159 (2′-O-Me) (FIG. 7D), the major organ of distribution was the kidney, with ARC159 showing the highest concentration by 3 hrs post-administration. Levels of [³H]-aptamer equivalents in the kidneys showed the following relation: [³H]-ARC159 (2′-O-Me)>[³H]-ARC158 (Arg₇)>[³H]-ARC83 (unconjugated) >[³H]-ARC120 (20 kDa PEG). Among all organs and tissues examined, highest mass-normalized concentrations of 20 kDa PEGylated aptamer (ARC120) were measured in the mediastinal lymph nodes rather than the kidneys (FIG. 6B).

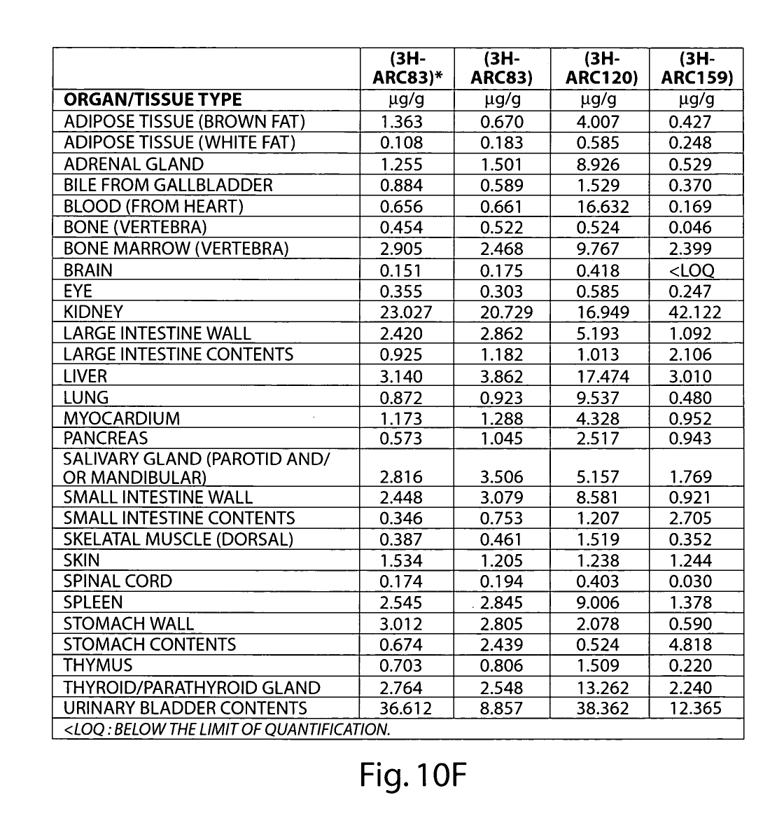

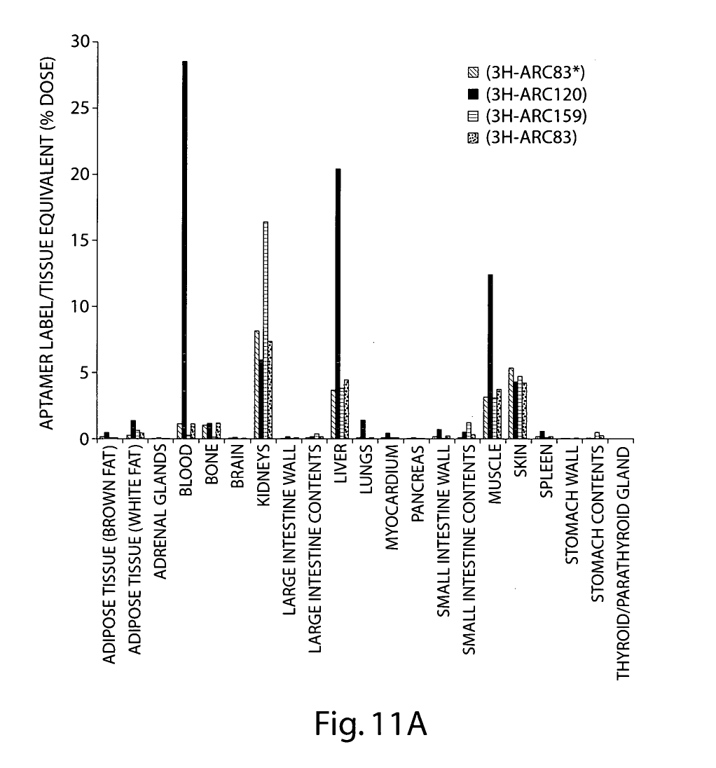

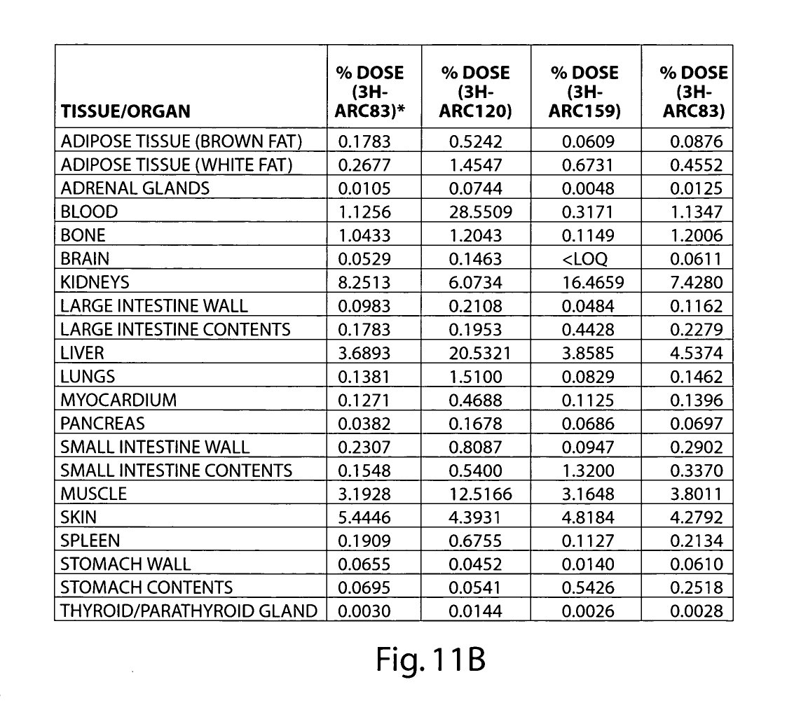

Urinary elimination of aptamers. Despite some variability between the two replicate animals dosed for each aptamer, total urinary output during the 24 hours following administration was generally similar. However, the timing and extent of urinary elimination differed among the aptamers. Total urinary elimination hovered between 20-30% of the administered dose for [³H]-ARC120 and [³H]-ARC158, 30-35% of the administered dose for [³H]-ARC83, and more than 40% of the administered dose for [³H]-ARC159 (FIG. 8). Consistent with these findings, intact [³H]-ARC159 was most abundant in the kidneys, with aptamers showing the relation: [³H]-ARC159 (2′-O-Me) >[³H]-ARC158 (Arg₇)>[³H]-ARC83 (unconjugated)>[³H]-ARC120 (20 kDa PEG) (FIG. 5). Analysis of urine samples using capillary gel electrophoresis (FIG. 9A) and MALDI-TOF (FIG. 9B) detected presence of full-length ARC159, but not other aptamers at 48 hrs (FIG. 9).

### Example 7

### Maximization of Targeted Tissue Distribution

An aptamer complexed to various PEG polymers of 20 kDa and 40 kDa molecular weight was generated as described in Example 1 and Tables 3A and 3B, supra. Following the pharmacokinetic studies described in Examples 4, 5 and 6, it was observed that the 20 kDa conjugate (ARC120) showed a significantly higher AUC value (i.e., the area under the concentration-time curve) relative to the 40 kDa PEG conjugate (ARC122), suggesting that the 20 kDa PEGylation may promote aptamer exposure to organs and tissues more effectively than 40 kDa PEGylation. Among the organs and tissues examined, the highest mass-normalized concentrations of 20 kDa PEGylated aptamer ARC120 were measured in the mediastinal lymph nodes rather than the kidneys, as shown in FIG. 7B. Though the main effect of conjugation to 20 kDa PEG was to retard renal filtration of aptamer, the comparatively high concentrations of 20 kDa PEG conjugate measured in well-perfused organs or tissues, such as inflamed tissue, relative to other aptamers or conjugates, demonstrates that PEGylation also promotes aptamer distribution to those particular tissues. The enhanced distribution of PEGylated aptamer to perfused organs represents extravasation, as suggested by results of autoradiography experiments in which [³H] signal was evident inside cells of both liver and kidney in mice dosed with tritiated ARC120 with a 20 kDa PEG conjugate. Thus, the methods of the present invention have been applied to direct a therapeutic aptamer conjugate to a particular tissue, a feature which enhances the therapeutic efficacy of the aptamer conjugate.

### Example 8

### Aptamer-Conjugate Pharmacokinetics and Biodistribution in Healthy and Inflamed Tissues

Aptamer synthesis. The ARC83 aptamer and its fully 2′-O-Me modified variant (ARC159) were synthesized as described in Example 1.

Synthesis of aptamer conjugates. The ARC83 aptamer was conjugated to a 20 kDa PEG moiety post-synthetically via amine-reactive chemistries to form the ARC120 aptamer. Conjugation was performed using the method described in Example 1. The composition of each aptamer conjugate was verified by polyacrylamide capillary gel electrophoresis, HPLC, and/or matrix-assisted laser desorption ionization time of flight mass spectrometry (MALDI-TOF). The aptamer conjugates were formulated for dosing after precipitation and desalting using CENTRISEP™ filters (Princeton Separations Inc., Adelphia, N.J.).

3H]-labeling of aptamers. The ARC83 and ARC159 aptamers were tritiated as described in Example 1. The tritiated 20 kDa PEG conjugate (ARC120) was prepared as described above on a small scale, using [³H]-ARC83 as starting material, followed by PAGE purification.

Dosing formulations. Radiolabeled aptamers were dissolved in 1× phosphate-buffered saline (PBS) (137 mM NaCl, 2.7 mM KCl, 10 mM NaHPO₄, 2 mM KH₂PO₄, pH 7.2) at a concentration of 1 mg/ml, as described above in Example 1. The specific activities for each of aptamer were as follows: ARC83: 25 μCi/mg; ARC120: 52 μCi/mg; and ARC159: 37 μCi/mg.

On the day prior to dosing, radiolabeled ARC83, ARC120 and ARC159 stock solutions were each diluted in 0.9% saline to a concentration of 0.357 mg/ml, to deliver a dose of 5 mg/kg (dosing volume of 14 mL/kg) and a target radioactivity level of approximately 100 μCi/animal. The amount of radioactivity in each formulation was verified by liquid scintillation counting. The overall mean radioactivity concentration (±S.D.) in the dose formulations were as follows: Groups 1 and 4: 710.7×106±5.1±106 dpm/g (320.13 μCi/g); Group 2: 708.3×106±5.5×10⁶ dpm/g (319.05 μCi/g); and Group 3: 900.3×106±16.1×106 dpm/g (405.55 μCi/g), with coefficient of variations (CV) ranging from 0.72 to 1.79%. Based on the standard deviations and the CVs around the mean concentration values, the dose formulations were considered homogeneous.

The doses of formulated [³H]-ARC83 administered to non-inflamed or inflamed animals by intravenous bolus injection were 4.57 mg/kg (114 μCi/non-inflamed animal) and 4.46 mg/kg (112 μCi/inflamed animal), respectively. The dose of formulated [³H]-ARC120 and [³H]-ARC159 administered were 4.77 mg/kg (114 μCi/non-inflamed animal) and 4.65 mg/kg (132 μCi/inflamed animal), respectively.

Animal procedures. Four week-old male CD-1 mice (25 g, n=6) were obtained from Charles River Canada, (Quebec, Canada). Animals were acclimated for one week, housed individually in stainless steel wire mesh-bottomed cages equipped with an automatic watering valve. Environmental conditions during the study were controlled at a target temperature and relative humidity of 22±3° C. and 50±20%, respectively. The photoperiod was 12 h light and 12 h dark. All animals had free access to a standard certified pelleted commercial laboratory diet (PMI Certified Rodent Chow 5002: PMI Nutrition International), except as indicated during designated procedures described below.

The radiolabeled [³H] aptamers were each administered to mice by intravenous bolus injection. For three animals, aptamer dosing occurred ten minutes following local treatment with carrageenan to induce paw inflammation as described below. All animals were euthanized by an intravenous injection of Euthanyl® (approximately 200 mg/kg) at 3 hours following administration of dose formulations.

Carrageenan-induced paw inflammation. Paw inflammation was induced in three of four animals by treatment with carrageenan. 10 ul of a 0.5% carrageenan solution (in 0.9% saline) was administered by insertion of a needle (29 ½-G) to a depth of approximately 1 mm, bevel down, through the callus of the right hind paw, at an angle nearly parallel with the foot pad. The left hind paw was not injected with carrageenan solution. 10 min following the administration of 0.5% carrageenan, each of the three treated animals was dosed with either formulated [³H]-ARC83, [³H]-ARC120, or [³H]-ARC159 by intravenous bolus injection as described above. Each injection was performed while the animal was in a restraining device through an ABBOCATH® that was flushed with 0.6 ml of saline after injection. In each case, inflammation was confirmed by the observation of redness and/or swelling. [³H]-ARC83 was also administered as described above to one control animal that did not undergo pre-treatment with carrageenan.

Quantitative Whole-Body Autoradiography (QWBA). Immediately following euthanasia, the right kidney and liver lateral lobe and caudate were removed from each animal and preserved in Bouin's fluid pending paraffin wax embedding and sectioning for micro-autoradiography. The remaining carcass was then closed/sutured and deep frozen in a mixture of hexane and dry ice for 10 min. Following the freezing procedure, the hind limbs were removed. The animal specimens were then embedded lying on their right sides in a 2% sodium carboxy methyl cellulose (CMC) medium using a freezing frame to collect sagittal whole-body sections. The hind limbs were also embedded in a lateral position in a 2% CMC medium, all at approximately the same level. Two blocks were prepared each containing 4 hind limbs. Holes were made in each frozen CMC block in order to incorporate ten [³H]-glucose standard solutions ranging from approximately 5 to 20000 nCi/g.

Each animal specimen block was sectioned using the Leica CM 3600 cryomicrotome. 30 μm sections were collected and identified. An appropriate number of anatomical levels were obtained to ensure inclusion of the following tissues for quantification:

From the animal body: adipose tissue, kidney, white fat, liver, brown fat, lung, adrenal gland, myocardium, bile from gallbladder, pancreas, blood from heart, salivary gland (parotid and/or mandibular), bone (vertebra), skeletal muscle (dorsal), bone marrow (vertebra), skin, brain, spinal cord, eye, spleen, gastrointestinal tract (GIT), thymus, stomach wall, thyroid/parathyroid gland, small intestine wall, urinary bladder content, large intestine wall, GIT contents (collected separately).

From the hind limb: bone, skin, bone marrow, tarsal-articular region, muscle/inflammatory region (paw), tendon fascia, muscle.

Micro-autoradiography. For each animal, at levels where the left kidney and remaining liver could be observed, six 10 μm sections containing both kidney and liver were collected and transferred to pre-treated 1″×3″ slides using the Macro-Tape Transfer System (MTTS) for micro-autoradiography. Six 10 μm sections containing the hind limbs were also collected and transferred to glass slides. Also, six 6 μm paraffin sections containing both kidney and liver were collected and de-parrafinated using standard procedures.

For the micro-autoradiography technique, glass slide sections (de-paraffinated glass slide sections and glass slide sections obtained from MTTS) were dipped in Kodak photographic emulsion NTB-2 and then exposed for a period of 7, 14 and 21 days in light-proof plastic boxes. Glass slide sections were then developed using the Kodak developer D-19 and Kodak fixer. Procedures were carried out under reduced safety light conditions whenever necessary. Following development, glass slide sections were stained and then examined using light microscopy. The localization and relative concentration of silver grains observed at the cellular surface were used to evaluate the distribution and the extent of radioactivity (semi-quantitative) in hind limbs (inflamed vs. non-inflamed tissue), kidney and liver.

For the quantitative autoradiography technique, sections collected were freeze-dried in the cryochamber for at least 16 hours prior to being exposed to an imaging plate for 96 h (whole-body sections) or 120 h (hind limb sections) in a lead box and refrigerated at 4° C. to minimize background radiation artifacts. Following exposure, the imaging plates were read by a Fuji BAS-2500 scanner and its Fuji Image Reader software version 1.1.

From the autoradiograms obtained, the amount of radioactivity in the above specified tissues were quantified from each animal with reference to the calibration curve generated by the known ³H-glucose standard solutions radioactivity concentrations using the Fuji Image Gauge Analysis software version 3.12. Each quantified result was corrected for background.

Quantitation of radioactivity. Concentration of radioactivity in whole-body tissues and hind limbs was determined by quantitative autoradiography. Tissue concentration of radioactivity (in nCi/g) was calculated in terms of mass eq/g on the basis of the calculated specific activity (dpm/mg) of the radiolabeled test article in the dose formulation. Numerical data obtained were subjected to calculation of group mean values and standard deviations. Evaluation of concentration of radioactivity at a cellular level was determined by micro-autoradiography (qualitative evaluation).

### Example 9

### Quantitative Whole-Body Tissue Distribution of Aptamers

The biodistribution of several aptamer compositions (i.e., the ARC83 unconjugated aptamer, the ARC120 20 kDa PEG conjugated aptamer, and the ARC159 fully 2′-O-Me modified aptamer, was assessed using quantitative whole-body autoradiography. Previous biodistribution studies, see, e.g. Healey et al.(2004) Pharm. Res., 21: 2234-2246, involving tissue harvesting, combustion, and liquid scintillation counting, analyzed distribution of radiolabeled aptamers to a small sampling of tissues. In this Example, the biodistribution of radiolabeled aptamers was tested in a large and diverse set of organs and tissues using the method of quantitative whole body radiography.

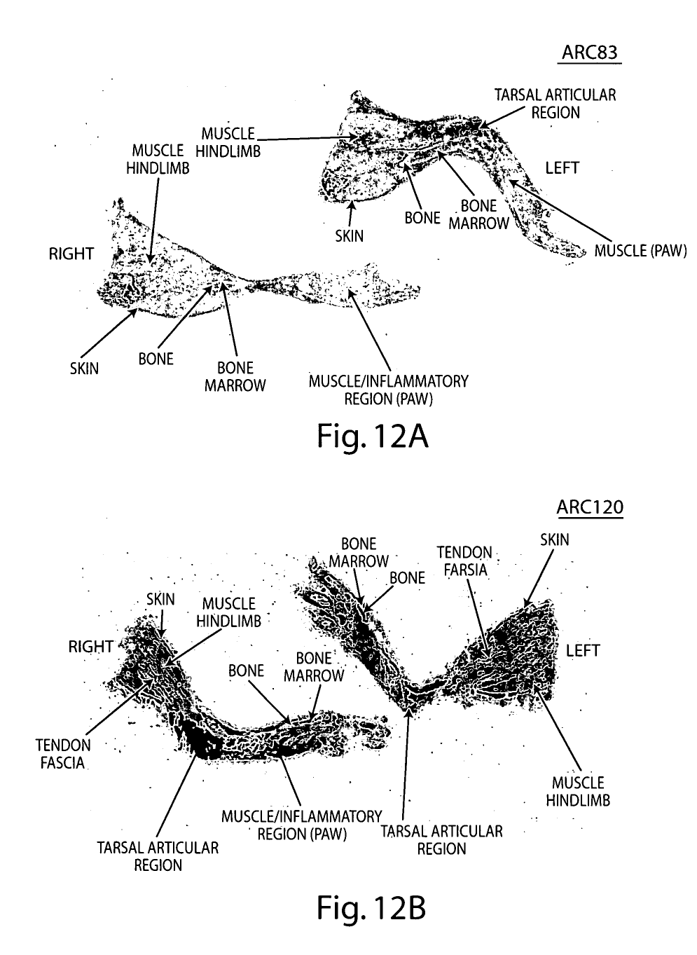

Mice were dosed with [³H]-labeled aptamers by intravenous bolus injection at a target dose level of 5 mg/kg (100 μCi/animal) as described in Example 8. The [³H]-ARC83 aptamer (the unconjugated aptamer), the [³H]-ARC120 aptamer (the ARC83 aptamer conjugated to a 120 kDa PEG), and the [³H]-ARC159 aptamer (the fully 2′-O-Me variant of the ARC83 aptamer) were administered 10 minutes after the right hind limbs of mice were treated with a solution of 0.5% carrageenan solution to induce localized paw inflammation. As described, animals were euthanized 3 hours post-dose, deep frozen and then carcasses and hind limbs were embedded and sectioned for determination of radiolabel-equivalent concentrations in organs and tissues by quantitative whole-body autoradiography. Representative autoradiographic images and concentrations of radioactivity in the analyzed whole-body tissues of individual animals are presented in FIG. 10. The [³H]-ARC83 aptamer was also administered to a healthy, control animal in which paw inflammation was not induced. Both animals presented relatively similar levels of radioactivity in whole-body tissues (FIGS. 10, A, D, E and F).

Highest concentrations of radiolabel in whole-body tissues for all animals were observed in the kidney and urinary bladder contents, which suggests that aptamer excretion occurs mainly via urine (FIGS. 10, A-F). Conversely, little radioactivity was generally associated with the brain, spinal cord and adipose tissue. While not intending to be bound by theory, these results may indicate that prolonged residence in the blood stream increases exposure of conjugated aptamer to tissues, thereby leading to enhanced uptake that is most pronounced in the case of highly perfused organs.

Overall, levels of radiolabel in whole-body tissues in the animal dosed with the [³H]-ARC120 (20 kDa PEG) aptamer were higher than the levels for either the [³H]-ARC83 (unconjugated) aptamer or the [³H]-ARC159 (fully 2′-O-Me) aptamer (FIGS. 10, B through E). Significantly higher concentrations of the [³H]-ARC120 equivalents relative to other aptamers were measured in blood, e.g., from the heart, and highly perfused organs or tissues, e.g., bone marrow, liver, lung, myocardium, spleen, and in several glandular tissues, e.g., adrenal, thyroid/parathyroid, salivary (FIGS. 10, B, E and F). The animal dosed with the [³H]-ARC159 (fully 2′-O-Me) aptamer showed the lowest whole-body levels of radiolabel in organs and tissues, but had the highest levels of radiolabel associated with the kidney and with the gastro-intestinal tract contents (FIGS. 10, C, E and F).

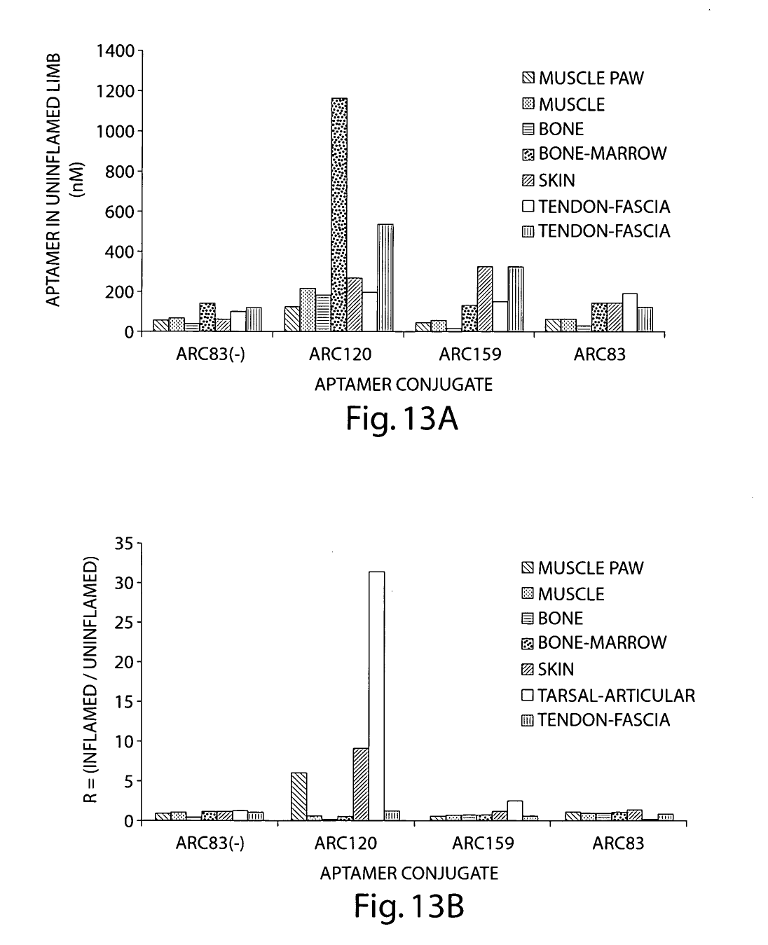

The different aptamers tested also showed varying patterns of whole-body distribution in terms of the amount of aptamer residing in particular organs or tissues as a percentage of the total administered dose (FIGS. 11A and B). The kidneys represented a major target organ for radiolabel, particularly in the case of ARC 159 (fully 2′-O-Me), where over 15% of the administered dose distributed to the kidneys. As expected due to the retarding effect of the 20 kDa PEG moiety on renal filtration, less of the administered dose of [³H]-ARC120 Oust over 5%) distributed to the kidneys and thus more remained in the vasculature, accessible to highly perfused organs such as liver. For all aptamers, primary sites of distribution included liver, kidney, muscle, and skin (FIGS. 11A and B) expressed as a percentage of the administered dose.

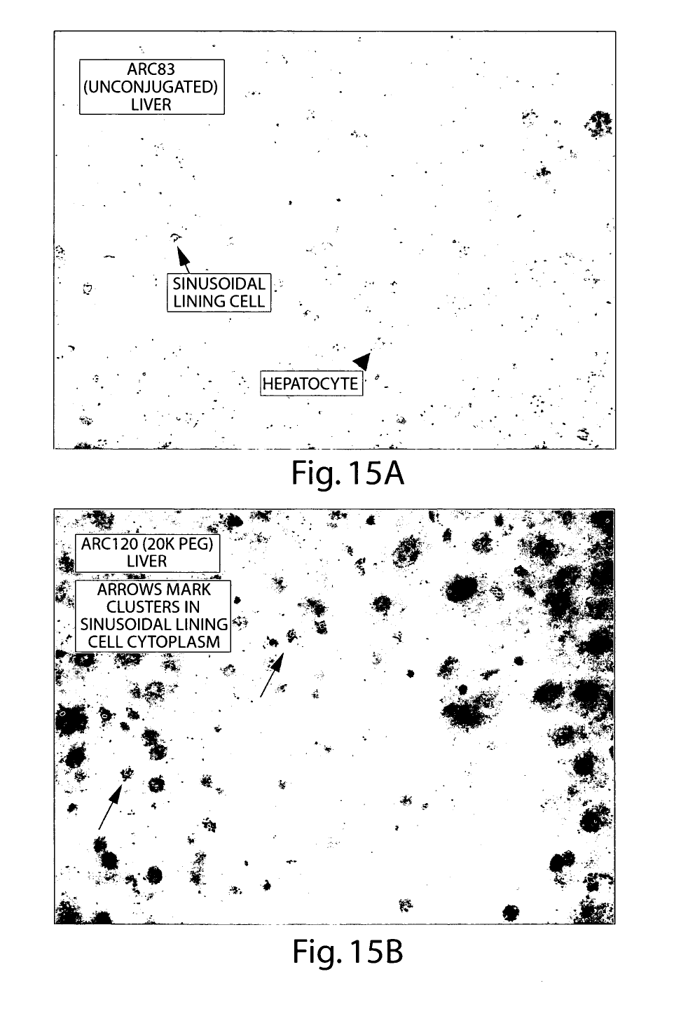

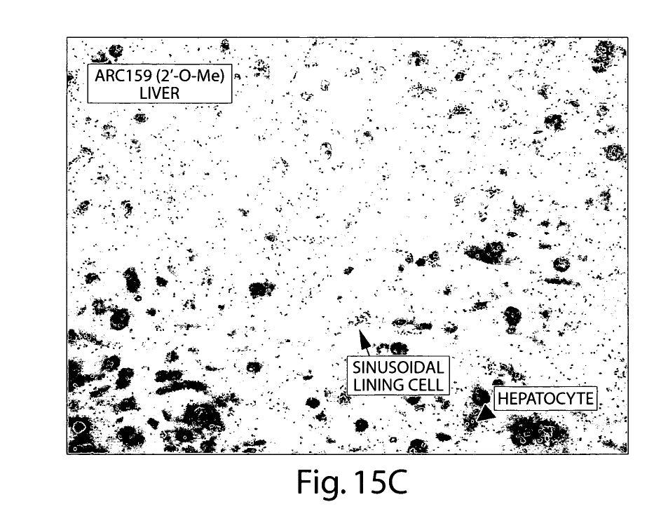

Presence of aptamer in residual blood may contribute to, but is unlikely to account entirely for, increased levels of the 20 kDa aptamer conjugate seen in perfused organs. The results of micro-autoradiography experiments in which [³H] signal is evident inside cell-types in both liver and kidney in mice dosed with tritiated ARC120 (20 kDa PEG) aptamer or other aptamers (described below in Example 11 and shown in FIGS. 14-15) suggest that the enhanced distribution of PEGylated aptamer to perfused organs actually represents extravasation.

The animal dosed with the [³H]-ARC159 fully 2′-O-Me composition aptamer had the lowest levels of whole-body radioactivity in tissues, but had the highest levels of radioactivity associated with the interior of the gastro-intestinal tract. As described above, ARC159 is rapidly eliminated from circulation, primarily by renal filtration. Compared to an aptamer composition in which the ARC83 aptamer contains a mixture of 2′-F and 2′-O-Me modified residues, the fully 2′-O-Me modified aptamer (i.e., ARC159) displayed very rapid loss from plasma and distribution into tissues, with the primary target organ being the kidney. Nonspecific protein-binding interactions are known to play an important role in the characteristic, rapid loss of phosphorothioate-containing antisense oligonucleotide from circulation and distribution to tissues. Thus, while not intending to be bound by theory, it is possible that the hydrophobic nature of the ARC159 (fully 2′-O-Me) aptamer may promote associations with factors that promote gastro-intestinal uptake or transport.

### Example 10

### Distribution of Aptamers to Inflamed Tissues

The ability of several aptamer compositions to access diseased tissues at sites of inflammation was assessed. Significant differences in aptamer localization to inflamed regions was observed.

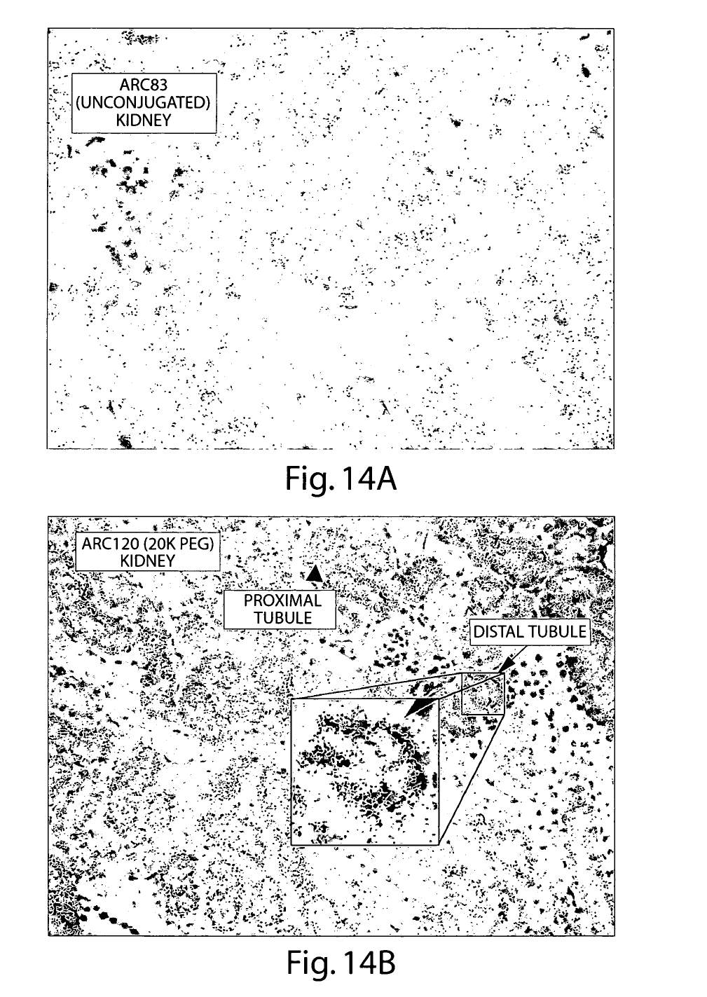

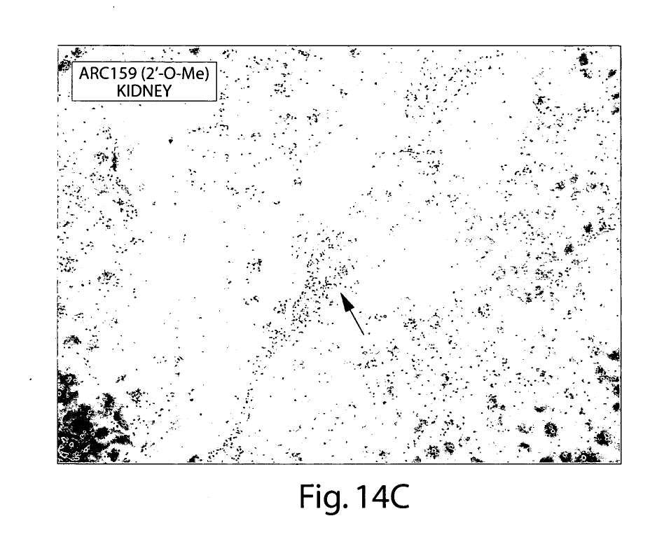

For all animals, relatively high concentrations of radioactivity were observed in the tarsal-articular region, bone marrow and tendon fascia, whereas comparatively low concentrations were observed in the bone (FIG. 12 and FIG. 13A). Generally, concentrations of radiolabel in hind limb tissues of the animal dosed with the [³H]-ARC120 (20 kDa PEG) aptamer (FIG. 12B) were higher than for animals dosed with the [³H]-ARC83 (unconjugated) aptamer (FIG. 12A) and the [³H]-ARC159 (fully 2′-O-Me) aptamer (not shown).

The 20 kDa PEG conjugate showed significantly greater distribution to the inflammation site relative to other aptamers (FIG. 13B). For the animal dosed with the [³H]-ARC120 (20 kDa PEG) aptamer, significant increases were observed in concentrations of radiolabel in several tissues of the inflamed hind limb, as compared to the non-inflamed hind limb, including the muscle/inflammatory region of the paw, skin, and the tarsal-articular region (FIGS. 12B and 13B). These differences were most pronounced in the case of the tarsal-articular region of the inflamed hind limb, which showed more than 30-fold higher levels of the [³H]-ARC120 (20 kDa PEG) aptamer than the non-inflamed hind limb from the same animal (FIGS. 12B and 13B).

Thus, this data demonstrates the capacity of PEGylation to facilitate aptamer access to inflamed tissues. Conjugation of an aptamer to a 20 kDa PEG moiety to retard renal filtration effectively promoted aptamer distribution to both healthy and inflamed tissues. The 20 kDa PEG conjugate (i.e., the [³H]-ARC120 aptamer) showed dramatically greater distribution of radioactivity to the inflammation site in carrageenan-treated animals than did other aptamers. In particular, significant increases, e.g., up to 30-fold, were seen in concentrations of radiolabel in the muscle/inflammatory region (paw), skin, tarsal-articular region and tendon fascia among non-inflamed and inflamed tissues in the animal dosed with the [³H]-ARC120 (20 kDa PEG) aptamer. These results indicate that, in addition to possessing desirable pharmacokinetic properties in vivo, PEGylated forms of aptamers directed against inflammatory disease targets are likely to achieve therapeutic dose levels in inflamed tissues. Thus, aptamers are capable of distributing to a wide variety of organs and tissues in vivo, and in some instances, to access the interior of cells, as described in Example 11 and shown in FIGS. 14-15.

### Example 11

### Cellular Distribution of Aptamers

Cellular distribution of radiolabeled aptamer equivalents was evaluated at a qualitative level by micro-autoradiography, as described above in Example 8. Cellular distribution of [³H] label was demonstrated in kidneys (FIGS. 14A-C) and liver (FIGS. 15A-C) in animals dosed with the [³H]-ARC83 (unconjugated), [³H]-ARC120 (120 kDa PEG), and [³H]-ARC159 (fully 2′-O-Me) aptamers. For all aptamers, clusters of silver grains were observed inside hepatocytes, sinusoidal lining cells, proximal and distal tubular cells, with somewhat different distribution patterns depending on the aptamer administered (FIGS. 14 and 15A-C). In the kidney, clear accumulation of [³H] label was seen in distal tubules in animals dosed with the [³H]-ARC120 (20 kDa PEG) aptamer conjugate, with an increased gradation in intensity from the proximal to distal direction (FIG. 14B). Similarly, clusters of silver grains were particularly pronounced in liver sinusoidal lining cells and hepatocytes in the animal dosed with the [³H]-ARC83 (unconjugated) aptamer (FIG. 15A).

The present invention having been described by detailed written description and the foregoing non-limiting examples, is now defined by the spirit and scope of the following claims.

1. A method of modulating in vivo aptamer distribution, comprising administering an aptamer composition to a subject wherein the chemical composition of the aptamer is formulated to modulate a pre-selected aptamer distribution property in vivo, and wherein, the pre-selected aptamer distribution property to be modulated is not reduction of aptamer clearance rate due to renal filtration.

2. The method of claim 1, wherein the pre-selected aptamer distribution property is preferential accumulation in a predetermined tissue or organ.

3. The method of claim 2, wherein the predetermined tissue or organ is a highly perfused tissue or organ.

4. The method of claim 3, wherein the aptamer comprises a nucleic acid sequence conjugated to a polyethylene glycol moiety.

5. The method of claim 4, wherein the highly perfused tissue or organ is selected from the group consisting of: kidney, liver, spleen, heart, lung and mediastinal lymph node.

6. The method of claim 2, wherein the predetermined tissue or organ is selected from the group consisting of: inflamed tissue, tumor tissue, and cancerous tissue.

7. The method of claim 4, wherein the polyethylene glycol moiety comprises a molecular weight selected from the group consisting of: 10, 20, 30, 40 and 60 kDa.

8. The method of claim 6, wherein the aptamer nucleic acid sequence binds specifically to a target that mediates allergic disease, inflammatory disease, rheumatoid arthritis, psoriasis or asthma.

9. The method of claim 8, wherein the aptamer nucleic acid binds specifically to a target that mediates inflammatory disease and the method further comprising administering the aptamer composition to a subject to treat or prevent inflammatory disease.

10. The method of claim 9, wherein the aptamer composition is administered systemically.

11. The method of claim 10, wherein the subject is human.

12. The method of claim 2, wherein the predetermined tissue or organ is the kidney or kidney tissue.

13. The method of claim 12, wherein the nucleic acid sequence of the aptamer is conjugated to a peptide.

14. The method of claim 13, wherein the peptide is selected from the group consisting of Ant, Tat and Arg₇.

15. The method of claim 1, wherein the aptamer increases the rate of aptamer clearance from the body.

16. The method of claim 15, wherein the nucleic acid sequence of the aptamer is conjugated to a moiety selected from the group consisting of Tat and cholesterol.

17. The method of claim 4, wherein the aptamer nucleic acid sequence binds specifically to a target that mediates cancer or infectious disease.

18. The method of claim 17, wherein the aptamer nucleic acid sequence binds specifically to a target that mediates cancer and the method further comprises administering the aptamer composition to a subject to treat or prevent cancer.

19. A method for treating a disease or disorder of a highly perfused tissue or organ comprising administering a PEGylated aptamer composition to a subject.

20. A method of treating or preventing cancer comprising administering PEGylated aptamer composition to a subject.

21. The method of claim 4, wherein the polyethylene glycol moiety comprises a molecular weight of no more than 20 kDa.

22. The method of claim 21, wherein the polyethylene glycol moiety comprises a molecular weight of no more than 10 kDa.


SEQUENCE LISTING (17 sequences)

SEQ ID NO: 1 - DNA, 93 bp, Artificial sequence
  synthetic template
catcgatgctagtcgtaacgatccnnnnnnnnnnnnnnnnnnnnnnnnnnnnnncgagaacgttctctcctctccctatagtgagtcgtatta

SEQ ID NO: 2 - DNA, 92 bp, artificial sequence
  synthetic template
catgcatcgcgactgactagccgnnnnnnnnnnnnnnnnnnnnnnnnnnnnnngtagaacgttctctcctctccctatagtgagtcgtatta

SEQ ID NO: 3 - DNA, 92 bp, artificial sequence
  synthetic template
catcgatcgatcgatcgacagcgnnnnnnnnnnnnnnnnnnnnnnnnnnnnnngtagaacgttctctcctctccctatagtgagtcgtatta

SEQ ID NO: 4 - DNA, 10 bp, bacteria
aacgttcgag

SEQ ID NO: 5 - DNA, 33 bp, artificial sequence
  synthetic aptamer
gggguuauuacagagucuguauagcuguaccct

SEQ ID NO: 6 - protein, 13 aa, HIV
RKKRRQRRRPPQC

SEQ ID NO: 7 - protein, 19 aa, Antennapedia
RQIKIWFQNRRMKWKKGGC

SEQ ID NO: 8 - protein, 8 aa, artificial sequence
  synthetic peptide
RRRRRRRC

SEQ ID NO: 9 - DNA, 33 bp, artificial sequence; identical to SEQ ID NO: 5
  synthetic aptamer

SEQ ID NO: 10 - DNA, 33 bp, artificial sequence; identical to SEQ ID NO: 5
  synthetic aptamer

SEQ ID NO: 11 - DNA, 33 bp, artificial sequence; identical to SEQ ID NO: 5
  synthetic aptamer

SEQ ID NO: 12 - DNA, 33 bp, artificial sequence; identical to SEQ ID NO: 5
  synthetic aptamer

SEQ ID NO: 13 - DNA, 33 bp, artificial sequence; identical to SEQ ID NO: 5
  synthetic aptamer

SEQ ID NO: 14 - DNA, 33 bp, artificial sequence; identical to SEQ ID NO: 5
  synthetic aptamer

SEQ ID NO: 15 - DNA, 35 bp, artificial sequence
  synthetic aptamer
ggagguuauuacagagucuguauagcuguacucct

SEQ ID NO: 16 - RNA, 16 nt, artificial sequence
  synthetic probe
acucuguaauaacccc

SEQ ID NO: 17 - RNA, 16 nt, artificial sequence
  synthetic probe
ggguacagcuauacag